(19) United States
(12) Patent Application Publication
(10) Pub. No.: US 2010/0298225 A1
(43) Pub. Date: Nov. 25, 2010

(54) PEPTIDE GAP JUNCTION MODULATORS

(76) Inventors: Bjarne Due Larsen, Roskilde (DK); Mario Delmar, Ann Arbor, MI (US); Steven M. Taffet, Syracuse, NY (US); Wanda Coombs, Syracuse, NY (US)

Correspondence Address: CLARK & ELBING LLP, 101 FEDERAL STREET, BOSTON, MA, 02110, US

(21) Appl. No.: 12/669,351

(22) PCT Filed: Jul. 14, 2008
(86) PCT No.: PCT/GB08/02405

§ 371 Date: May 27, 2010

Related U.S. Application Data
(60) Provisional application No. 60/959,650, filed on Jul. 16, 2007.

(30) Foreign Application Priority Data
Jul. 15, 2007 (DK) PA 2007 01044

(51) Int. Cl. A61K 38/16; C07K 14/00; C07K 7/06; A61P 9/00; C07K 7/08; A61P 29/00; A61K 38/10; A61K 38/08; A61P 27/16; A61P 13/10; A61P 13/12; A61P 25/00; A61P 37/06; A61P 35/00; A61P 11/00

(52) U.S. Cl. 514/15.4; 530/326; 530/328; 530/330; 530/327; 530/324; 514/21.3; 514/21.5; 514/21.8; 514/21.6; 514/21.4; 514/19.3; 514/18.3; 514/17.7

(57) ABSTRACT

Disclosed are peptides that facilitate the intercellular communication mediated by gap junctions. The invention has a wide spectrum of useful applications including use in the treatment of diseases associated with impaired gap junction intracellular communication (GJIC).

### FIELD OF THE INVENTION

The invention relates to peptides capable of modulating intracellular gap junctional communication. The invention also relates to methods of using the peptides to modulate such communication, to the use of the peptides for the manufacture of medicaments for the prevention and/or treatment of conditions associated with said communication and to pharmaceutical compositions comprising said dipeptides.

### BACKGROUND

There is increasing recognition that intercellular communication is essential for cellular homeostasis, proliferation and differentiation. Such communication is believed to be facilitated by gap junctions. These structures are thought to be a route for coupling cells and permitting “cross-talk”. See generally, Sperelakis, N., (1989) Cell Interactions and Gap Junctions by N. Sperelakis, William C. Cole (Editor).

Connexins are integral membrane proteins that oligomerize to form intercellular channels called gap junctions. The most abundant gap junction protein in a number of mammalian systems is connexin43 (“Cx43”).

Gap junction channels are responsible for direct cell-to-cell communication. These channels are dynamic pores that are regulated in response to changes in the cellular environment and by protein interactions. In the heart, gap junction channels are a critical mechanism for the passage of electrical impulses (Lerner et al., “Accelerated Onset and Increased Incidence of Ventricular Arrhythmias Induced by Ischemia in Cx43-deficient Mice,” Circ. 101(5):547-552 (2000); Gutstein et al., “Conditional Gene Targeting of Connexin43: Exploring the Consequences of Gap Junction Remodeling in the Heart,” Cell Commun. Adhes. 8(4-6):345-348 (2001); Vaidya et al., “Null Mutation of Connexin43 Causes Slow Propagation of Ventricular Activation in the Late Stages of Mouse Embryonic Development,” Circ. Res. 88(11):1196-1202 (2001)). Each channel is composed of two identical hexameric structures or connexons that dock across an extracellular space. The result is a permeable pore that is dynamically regulated. The individual subunit of the connexon is the molecule connexin. This molecule resides in the membrane, with its N-terminal (“NT”), cytoplasmic loop (“CL”) and C-terminal (“CT”) domains in the cytoplasmic space, as illustrated in FIG. 1. In addition, there are four transmembrane domains and two extracellular domains (extracellular loops) that are involved in the docking to the opposing connexon. There are at least 20 different connexin isotypes in the mouse genome and 21 in the human genome (Willecke et al., “Structural and Functional Diversity of Connexin Genes in the Mouse and Human Genome,” Biol. Chem. 383(5):725-737 (2002)). The most abundant connexin isotype in the heart, brain and other tissues is the 43 KDa protein, Cx43. Gap junctions allow the passage of ions and small molecules between cells and are regulated by a variety of chemical interactions between the connexin molecule and the microenvironment. As such, gap junctions act as active filters to control the passage of intercellular messages to modulate function.

Previous work has suggested that regulation of Cx43 channels results from the association of the CT domain, acting as a gating particle, and a separate region of the connexin molecule acting as a receptor for the gating particle (Duffy et al., “pH-Dependent Intramolecular Binding and Structure Involving Cx43 Cytoplasmic Domains,” J. Biol. Chem. 277(39):36706-36714 (2002); Moreno et al., “Role of the Carboxyl Terminal of Connexin43 in Transjunctional Fast Voltage Gating,” Circ. Res. 90(4):450-457 (2002), 92(1):e30 (2003) (erratum)). Additional studies have shown that this intra-molecular interaction can be modulated by other inter-molecular interactions in the microenvironment of the gap junction plaque (Morley et al., “Intramolecular Interactions Mediate pH Regulation of Connexin43 Channels,” Biophys. J. 70(3):1294-1302 (1996)). Thus, the emerging picture of a Cx43 gap junction plaque is that of a macromolecular complex where proteins act in concert to modulate intercellular communication. At the center of these interactions is the CT domain, which acts as a substrate for a number of kinases (Duffy et al., “Regulation of Connexin43 Protein Complexes by Intracellular Acidification,” Circ. Res. 94(2):215-222 (2004); Giepmans et al., “Interaction of c-Src with gap junction protein connexin-43. Role in the Regulation of Cell-cell Communication,” J. Biol. Chem. 276(11):8544-8549 (2001); Kanemitsu et al., “Tyrosine Phosphorylation of Connexin 43 by v-Src is Mediated by SH2 and SH3 Domain Interactions,” J. Biol. Chem. 272(36):22824-22831 (1997); Lampe et al., “Phosphorylation of Connexin43 on Serine-368 by Protein Kinase C Regulates Gap Junctional Communication,” J. Cell Biol. 149(7):1503-1512 (2000); Lau et al., “Regulation of Connexin43 Function by Activated Tyrosine Protein Kinases,” J. Bioenerg. Biomembr. 28(4):359-368 (1996); Shin et al., “The Regulatory Role of the C-Terminal Domain of Connexin43,” Cell Commun. Adhes. 8(4-6):271-275 (2001); TenBroek et al., “Ser364 of Connexin43 and the Upregulation of Gap Junction Assembly by cAMP,” J. Cell Biol. 155(7):1307-1318 (2001)), a ligand for noncatalytic proteins (Giepmans & Moolenaar, “The Gap Junction Protein Connexin43 Interacts with the Second PDZ Domain of the Zona Occludens-1 Protein,” Curr. Biol. 8(16):931-934 (1998); Giepmans et al., “Connexin-43 Interactions with ZO-1 and Alpha- and Beta-tubulin,” Cell Commun. Adhes. 8(4-6):219-223 (2001); Toyofuku et al., “Direct Association of the Gap Junction Protein Connexin-43 with ZO-1 in Cardiac Myocytes,” J. Biol. Chem. 273(21):12725-12731 (1998); Toyofuku et al., “c-Src Regulates the Interaction between Connexin-43 and ZO-1 in Cardiac Myocytes,” J. Biol. Chem. 276(3):1780-1788 (2000); Zhou et al., “Dissection of the Molecular Basis of pp 60(v-Src) Induced Gating of Connexin 43 Gap Junction Channels,” J. Cell Biol. 144(5):1033-1045 (1999); Ai et al., “Wnt-1 Regulation of Connexin43 in Cardiac Myocytes,” J. Clin. Invest. 105(2):161-171 (2000); Xu et al., “N-Cadherin and Cx43a1 Gap Junctions Modulates Mouse Neural Crest Cell Motility Via Distinct Pathways,” Cell Adhes. Commun. 8(4-6):321-324 (2001); Schubert et al., “Connexin Family Members Target to Lipid Raft Domains and Interact with Caveolin-1,” Biochem. 41(18):5754-5764 (2002)), and a gating particle to modify coupling between cells (Duffy et al., “pH-Dependent Intramolecular Binding and Structure Involving Cx43 Cytoplasmic Domains,” J. Biol. Chem. 277(39):36706-36714 (2002); Moreno et al., “Role of the Carboxyl Terminal of Connexin43 in Transjunctional Fast Voltage Gating,” Circ. Res. 90(4):450-457 (2002), 92(1):e30 (2003) (erratum); Morley et al., “Intramolecular Interactions Mediate pH Regulation of Connexin43 Channels,” Biophys. J. 70(3):1294-1302 (1996); Anumonwo et al., “The Carboxyl Terminal Domain Regulates the Unitary Conductance and Voltage Dependence of Connexin40 Gap Junction Channels,” Circ. Res. 88(7):666-673 (2001)).

The pharmacology of gap junctions has been reviewed by Srinivas et al. (Srinivas et al., “Prospects for Pharmacological Targeting of Gap Junction Channels,” in CARDIAC ELECTROPHYSIOLOGY: FROM CELL TO BEDSIDE 158-167 (Douglas Zipes & Jose Jalife eds., 4th ed. 2004)). There are few specific drugs for the modulation of gap junction function. Long chain alkanols (heptanol and octanol) have long been used as uncoupling agents. The mechanism of action is unknown although membrane fluidity is thought to be the primary target. General anesthetics have also been shown to uncouple gap junctions (Burt & Spray, “Volatile Anesthetics Block Intercellular Communication between Neonatal Rat Myocardial Cells,” Circ. Res. 65(3):829-837 (1989)), but, again, the mechanism is unknown and not specific to gap junction proteins. Some agents such as butanedione monoxime (Nerve & Sarrouilhe, “Modulation of Junctional Communication by Phosphorylation: Protein Phosphatases, the Missing Link in the Chain,” Biol. Cell 94(7-8):423-432 (2002)) are thought to modify phosphorylation of gap junctions leading to uncoupling. Flufenamic acid has been shown to be an effective inhibitor of gap junctions but the mechanism is thought to be indirect and not specific to any connexin protein (Srinivas & Spray, “Closure of Gap Junction Channels by Arylaminobenzoates,” Mol. Pharmacol. 63(6):1389-1397 (2003)).

Peptides have been shown to alter the function of gap junction channels. Antiarrhythmic peptide 10 (“AAP10”) alters gap junctional communication (Muller et al., “Actions of the Antiarrhythmic Peptide AAP10 on Intercellular Coupling,” Naunyn Schmiedebergs Arch. Pharmacol. 356(1):76-82 (1997); Dhein et al., “Effects of the New Antiarrhythmic Peptide ZP123 on Epicardial Activation and Repolarization Pattern,” Cell Commun. Adhes. 10(4-6):371-378 (2003)) indirectly by interaction with a membrane receptor and possibly by a PKC dependent mechanism (Dhein et al., “Protein Kinase Cα Mediates the Effect of Antiarrhythmic Peptide on Gap Junction Conductance,” Cell Commun. Adhes. 8(4-6):257-264 (2001)) which would lead to a variety of alterations in the target cell. Specific inhibition of gap junction formation has been demonstrated with the use of extracellular loop peptides (Kwak & Jongsma, “Selective Inhibition of Gap Junction Channel Activity by Synthetic Peptides,” J. Physiol. 516(3):679-685 (1999)). It is thought that these peptides inhibit gap junctions by preventing connexon docking in the extracellular gap. These effects are slow and the interaction requires high concentrations of peptide (Dahl et al., “Attempts to Define Functional Domains of Gap Junction Proteins with Synthetic Peptides,” Biophys. J. 67(5):1816-1822 (1994)).

What is needed is a strategy to use structural, binding and functional assays to develop a peptide-based approach to Cx43 regulation; and agents that can maintain gap junction channels in an open state.

The present invention is directed to overcoming these deficiencies in the art.

### SUMMARY OF THE INVENTION

The invention generally relates to peptides that modulate gap junction intercellular communication (GJIC). The invention has a wide spectrum of useful applications including use in the treatment or prevention of pathologies associated with impaired GJIC.

In a first aspect of the present invention, peptides according to Formula I and pharmaceutically acceptable salts thereof are provided:

### Formula I:

R¹—Z—(L-Q)ₚ-R²

wherein
R¹ is selected from H, Ac, benzoyl and Tfa;
Z is A1-A2-A3-A4 or a retro analogue thereof, wherein:
A1 is a basic amino acid such as Arg, Lys or H is or A1 is a lysine mimetic;
A2 is a basic amino acid such as Arg, Lys or H is or A2 is a lysine mimetic;
A3 is any amino acid, preferably selected from Gly, Pro, Ala, Val, Leu, Ile, Met, Cys, Phe, Tyr, Trp, H is, Lys, Arg, Gln, Asn, Glu, Asp, Ser and Thr, which amino acid is optionally modified with B, or A3 is a lysine mimetic, which lysine mimetic is optionally modified with B, or A3 is Glx(CONH-B);
A4 is an aromatic amino acid such as Trp, Tyr, Phe or H is, or A4 is an aliphatic amino acid such as Ala, Val, Leu or Ile, or A4 is Met, or A4 is missing.
wherein B is a hydrophobic group
   L is X1-X2-X3-X4-X5-X6-X7-X8-X9-X10-X1′-X12-X13-X14-X15 or a retro analogue thereof, wherein
X1 is Gly, Ala, Ser, 8-amino-3,6-dioxaoctanic acid or is missing;
X2 is Gly, Ala, Ser, or is missing;
X3 is Gly, Ala, Ser, Leu, Val, Ile or is missing;
X4 is Gly, Ala, Ser, or is missing;
X5 is Gly, Ala, Ser, Arg, Lys, H is or is missing;
X6 is Val, Ile, Leu, Gly, Ala, Ser, or is missing;
X7 is Pro, Gly, Ala, Ser, or is missing;
X8 is Trp, Tyr, Phe, Leu, Val, Ile or is missing;
X9 is Trp, Tyr, Phe, Gly, Ala, Ser, or is missing;
X10 is Gly, Ala, Ser, Arg, Lys, H is or is missing;
X11 is Arg, Lys, H is, Gly, Ala, Ser, or is missing;
X12 is Gly, Ala, Ser, or is missing;
X13 is Val, Leu, Ile or is missing;
X14 is Gly, Ala, Ser, or is missing;
X15 is Arg, Lys, H is or is missing;
   Q is A5-A6-A7-A8, or a retro analogue thereof, wherein
A5 is a basic amino acid such as Arg, Lys or H is or A5 is a lysine mimetic;
A6 is a basic amino acid such as Arg, Lys or H is or A6 is a lysine mimetic;
A7 is any amino acid, preferably selected from Gly, Pro, Ala, Val, Leu, Ile, Met, Cys, Phe, Tyr, Trp, H is, Lys, Arg, Gln, Asn, Glu, Asp, Ser and Thr, which amino acid is optionally modified with B, or A7 is a lysine mimetic, which lysine mimetic is optionally modified with B, or A7 is Glx(CONH-B);
A8 is an aromatic amino acid such as Trp, Tyr, Phe or H is, or A8 is an aliphatic amino acid such as Ala, Val, Leu or Ile, or A8 is Met, or A8 is missing.

### R² is NH₂, OH, OR, NHR, NRR

wherein R is 01-06 alkyl; and
   p is 0, 1, 2, 3, 4 or 5.

In some preferred embodiments of the first aspect of the invention, p=1 and accordingly the peptides of the present invention are represented by Formula II:

### Formula II:

R¹—Z-L-Q-R²

wherein Z, L, Q, R¹ and R² are as defined above for Formula I.

In other preferred embodiments of the first aspect of the invention, p=0 and the peptides of the present invention are represented by Formula III.

### Formula III:

R¹—Z—R²

wherein Z, L, Q, R¹ and R² are as defined above for Formula I.

The peptides of the present invention may also comprise a peptide bond that is alkylated or otherwise modified to stabilize the peptide against enzymatic degradation and/or may comprise D-amino acids.

Peptides within the scope of the present invention are in one embodiment represented herein with free N-terminal and/or C-terminal group. These groups may remain free for some invention uses. However, in another embodiment, the peptides can feature blocked C-terminal groups and free N-groups. Alternatively, such peptides may have blocked N-groups and free C-terminal groups, or blocked N- and C-terminal groups. The nature of the terminal groups at either side of the molecule is not critical.

Additionally, amino acid residues within the peptides may be D- or L-amino acids. In certain aspects, to enhance stability of the compounds, D- amino acids may be preferred.

The peptides according to the invention have a wide variety of important uses and advantages.

For instance, such peptides may be used for preventing and/or treating conditions associated with impaired gap junction function resulting in reduced intercellular communication or overcoupling of intercellular communication or misregulated cellular communication.

Accordingly, in one aspect the present invention provides peptides as described herein for use in methods of medical treatment. Preferably, the method of medical treatment is treating a pathological condition involving impaired gap junctional communication.

Another aspect of the invention relates to the manufacture of a medicament for preventing and/or treating conditions associated with impaired gap junction function.

In another aspect, the invention provides a method of medical treatment, comprising administering to a patient having, or at risk of developing such a condition, a therapeutically effective amount of any of the peptides described above. In one preferred aspect, a patient is a human being.

Examples of conditions which can be treated include, but are not limited to, cardiovascular disease, inflammation of airway epithelium, disorders of alveolar tissue, bladder incontinence, impaired hearing due to diseases of the cochlea, endothelial lesions, diabetic retinopathy and diabetic neuropathy, ischemia of the central nervous system and spinal cord, dental tissue disorders including periodontal disease, kidney diseases, failures of bone marrow transplantation, wounds, erectile dysfunction, urinary bladder incontinence, neuropathic pain, subchronic and chronic inflammation, cancer and failures of bone marrow and stem cell transplantation, conditions which arise during transplantation of cells and tissues or during medical procedures such as surgery; as well as conditions caused by an excess of reactive oxygen species and/or free radicals and/or nitric oxide. Further examples of conditions which can treated include psoriasis, osteroporosis and diabetes.

A further aspect of the invention is pharmaceutical compositions, comprising any of the peptides described herein and a pharmaceutically acceptable carrier. Preferably, the pharmaceutical compositions are suitable for use in the methods of medical treatment described above. Preferably, the carrier is sterile, pyrogen-free and virus-free.

In the following sections, embodiments and examples of the invention will be described with reference to the figures. The embodiments and examples described here in are provided for illustrative purposes and should not be construed as limiting the scope of the invention.

### BRIEF DESCRIPTION OF THE DRAWINGS

FIGS. 1A-C are graphs of junctional current (Iⱼ) traces obtained from Cx43-expressing N2a cell pairs before and during perfusion with 1,5 mM octanol (onset indicated by thick vertical arrow) (transjunctional voltage: 60 mV; pulse duration: 10 seconds; interpulse interval: 10 seconds). In FIG. 1A, patch pipettes were filled with normal internal pipette solution. In FIG. 1B, the pipette solution contained peptide #2371 at a concentration of 0.1 mM and in FIG. 10, the peptide solution contained peptide 2372. The presence of either peptide 2371 or peptide 2372 in the patch pipette prevented octanol-induced uncoupling.

FIGS. 2A-F are graphs of the time course of octanol-induced changes in coupling recorded from Cx43-expressing N2a cells. Experiments were conducted in the absence (N=7) or in the presence (N=7) of 0.1 mM of the peptides 2366, 2371, 2372 and 2497 in the internal pipette solution. Time zero corresponds to the onset of octanol superfusion.

“Uncoupling” refers to the complete loss of junctional current under the voltage clamp protocol illustrated in FIGS. 1A-C and described in example 7.

FIGS. 2A,C and E illustrate the percent of pairs that remained coupled at the end of each minute after onset of octanol. FIGS. 2B, D and F shows the average junctional conductance (Gⱼ) as a function of time after onset of octanol. For each cell pair, Gⱼ was measured relative to the value recorded before octanol exposure. It can be seen for the peptides 2371(2A-B), 2372(2A-B) and 2366(20-0), that the average Gj decreased at first and then increased. The latter is reflective of the ability of these peptides to re-open gap junctions that were initially closed by the uncoupler. The peptide 2497(2E-F) is preventing the uncoupling of GJ.

FIGS. 3A-E are graphs of the time course of octanol-induced changes in coupling recorded from Cx43-expressing N2a cells. Dual cell patch clamp experiments were conducted in the absence (black trace; square data points) or in the presence (red trace; round data points) of 0.1 mM of the peptides 2517, 2518, 2519, 2520 and 2624 in the internal pipette solution. Time zero corresponds to the onset of octanol superfusion.

### DETAILED DESCRIPTION OF THE INVENTION

As discussed above, the invention relates to peptides that modulate gap junction intercellular communication (GJIC). The invention has a wide spectrum of useful applications including use in the treatment or prevention of pathologies associated with impaired GJIC.

The peptides of the present invention are represented by Formula I, as described above.

In embodiments wherein A1 is a basic amino acid, preferably it is Lys or Arg. In embodiments wherein A1 is a lysine mimetic, preferably it is (4S,2R) Amp. In embodiments wherein A2 is a basic amino acid, preferably it is Lys or Arg. In embodiments wherein A2 is a lysine mimetic, preferably it is (4S,2R) Amp(Ac). In some embodiments, it is preferred that A1 and A2 are the same amino acid, or the same lysine mimetic. For example, A1 and A2 may both be Arg, or may both be Lys, or may both be (4S,2R)Amp. It is particularly preferred that A1 and A2 are the same when p=0 and/or when A4 is missing.

When A4 is present, A3 is preferably an amino acid selected from Gly, Pro, Ala, Val, Leu, Ile, Met, Cys, Phe, Tyr, Trp, His, Lys, Arg, Gln, Asn, Glu, Asp, Ser and Thr, more preferably Asn or Gln. When p=0 and A4 is present, it is preferable that A3 is Asn or Gln.

When A4 is missing, A3 is preferably an amino acid selected from Gly, Pro, Ala, Val, Leu, Ile, Met, Cys, Phe, Tyr, Trp, His, Lys, Arg, Gln, Asn, Glu, Asp, Ser and Thr, which amino acid is modified with B, or a lysine mimetic modified with B or GIx(CONH-B), more preferably Lys(NH-B), Asn(CONH-B), Glx(CONH-B) or a lysine mimetic modified with B. A3 may be

Lys(NH-B), Glx(CONH-B) or a lysine mimetic modified with B. For example, A3 may be Lys(4-hydroxybenzoyl) or Asn(benzyl).

In some embodiments, A4 is preferably an aromatic amino acid such as Trp, Tyr, Phe or H is or A4 is missing. More preferably, A4 is Trp, Tyr, Phe or is missing. Most preferably, A4 is Tyr or missing. In some embodiments wherein p=0, it may be preferred that A4 is an aliphatic amino acid such as Ala, Val, Leu or Ile, or the amino acid Met.

The peptides may comprise a retro analogue of Z, i.e. wherein Z is A4-A3-A2-A1.

As described above, B is a hydrophobic group. Preferably, B is a hydrophobic group comprising an optionally substituted aromatic carbon ring, preferably a 6- or 12-membered carbon aromatic ring. B may be optionally substituted as described below. Preferably B is an optionally substituted aralkyl, aryl or aroyl group, such as benzyl, hydroxybenzyl, phenyl, hydroxyphenyl, naphthyl, hydroxynaphthyl, benzoyl, hydroxybenzoyl, naphthoyl or hydroxynaphthoyl. Most preferably, B is benzyl, 4-hydroxybenzyl, benzoyl or 4-hydroxybenzoyl.

When an amino acid residue of the present invention is modified with B, preferably B is attached to the amino acid residue by a covalent bond. More preferably, B is attached by a covalent bond to the side chain of the amino acid residue. Most preferably, B is attached to the end of the side chain distal from the peptide backbone.

In embodiments wherein p is not 0, Q is present. The peptides may comprise a retro analogue of Q, i.e. wherein Q is A8-A7-A6-A5.

When A5 is a basic amino acid, preferably it is Lys or Arg. When A5 is a lysine mimetic, preferably it is (4S,2R) Amp. When A6 is a basic amino acid, preferably it is Lys or Arg. When A6 is a lysine mimetic, preferably it is (4S,2R) Amp(Ac). In some embodiments, it is preferred that A5 and A6 are the same amino acid, or the same lysine mimetic. For example, A5 and A6 may both be Arg, or may both be Lys, or may both be (4S,2R)Amp.

When A8 is present, A7 is preferably an amino acid selected from Gly, Pro, Ala, Val, Leu, Ile, Met, Cys, Phe, Tyr, Trp, His, Lys, Arg, Gin, Asn, Glu, Asp, Ser and Thr, more preferably Asn or Gln.

When A8 is missing, A7 is preferably an amino acid selected from Gly, Pro, Ala, Val, Leu, Ile, Met, Cys, Phe, Tyr, Trp, His, Lys, Arg, Gln, Asn, Glu, Asp, Ser and Thr, which amino acid is modified with B, or a lysine mimetic modified with B or Glx(CONH-B), more preferably Lys(NH-B), Asn(CONH-B), Glx(CONH-B) or a lysine mimetic modified with B. A7 may be Lys(NH-B), Glx(CONH-B) or a lysine mimetic modified with B. For example, A7 may be Lys(4-hydroxybenzoyl) or Asn(benzyl).

In some embodiments, A8 is preferably an aromatic amino acid such as Trp, Tyr, Phe or His or A8 is missing. More preferably, A8 is Trp, Tyr, Phe or is missing. Most preferably, A8 is Tyr or missing. In some embodiments wherein p=0, it may be preferred that A8 is an aliphatic amino acid such as Ala, Val, Leu or Ile, or the amino acid Met.

It may be preferable that A1 and A5 are the same amino acid or the same lysine mimetic, and/or that A2 and A6 are the same amino acid or lysine mimetic, and/or that A3 and A7 are the same amino acid or lysine mimetic, or are both Glx(NH-B), and/or that A4 and A8 are the same amino acid or are both missing.

In some embodiments wherein p is not 0, it is preferred that L is absent, i.e. that each of X1 to X15 is missing.

Preferably, p is 0 or 1, more preferably 0.

Preferably R¹ is Ac. Preferably R² is NH₂. In some preferred embodiments, R¹ is Ac and

R² is NH₂.

A first subset of peptides of the present invention are peptides according to Formula III, i.e. p=0, wherein

### A¹ is Lys, Arg or (2S4R)Amp;

### A2 is Lys, Arg or (2S4R)Amp(Ac);

A3 is Lys(NH-B) or Asn(CONH-B) if A4 is missing, and A3 is Asn or Gln if A4 is present; and
A4 is Trp, Tyr, Phe or His or is missing.

It is preferable in said first subset of peptides of the present invention that A1 and A2 are the same, i.e. A1 and A2 are both Lys, both Arg or both Amp. It is also preferable in said first subset that A3 is Lys(4-hydroxybenzoyl) or Asn(benzyl) if A4 is missing, and that A3 is Asn if A4 is present. It is also preferable in said first subset that A4 is Tyr or missing. It is also preferable in said first subset that R¹ is Ac and R² is NH₂.

A second subset of peptides of the present invention are peptides wherein p=1, 2, 3, 4 or 5, and wherein

A1 and A5 are the same amino acid or the same lysine mimetic;
A2 and A6 are the same amino acid or lysine mimetic;
A3 and A7 are the same amino acid or lysine mimetic, or are both GIx(NH-B);
A4 and A8 are the same amino acid or are both missing.

More preferable peptides within the second subset have Formula II, i.e. p=1. It is preferable in said second subset that A1 and A5, and A2 and A6 are all the same amino acid or lysine mimetic. It is also preferable in said second subset that A1 and A5 are both Arg. It is also preferable in said second subset that A2 and A6 are both Arg. It is also preferable in said second subset that A3 and A7 are both Asn. It is also preferable in said second subset that A4 and A8 are both Tyr.

Examples of compounds according to the invention include:

### Ac-Lys-Lys-Asn-Phe-NH₂

### Ac-Lys-Lys-Asn-Tyr-NH₂

### Ac-Lys-Lys-Asn-His -NH₂

### Ac-Lys-Lys-Asn-Trp-NH₂

### Ac-Lys-Lys-Asn-Val-NH₂

### Ac-Lys-Lys-Asn-Leu-NH₂

### Ac-Lys-Lys-Asn-Ile-NH₂

### Ac-Lys-Lys-Asn-Met-NH₂

### Ac-Arg-Arg-Asn-His-NH₂

### Ac-Arg-Arg-Asn-Trp-NH₂

### Ac-Arg-Arg-Asn-Ile-NH₂

### Ac-Arg-Arg-Asn-Met-NH₂

### Ac-Arg-Arg-Asn-Tyr-NH₂

### Ac-Arg-Arg-Asn-Val-NH₂

### Ac-Arg-Arg-Asn-Leu-NH₂

### Ac-Arg-Arg-Asn-Phe-NH₂

### Ac-(D-Arg)-(D-Arg)-Asn-Tyr-NH₂

### Ac-(D-Arg)-(D-Arg)-Asn-His-NH₂

### Ac-(D-Arg)-(D-Arg)-Asn-Trp-NH₂

### Ac-(D-Arg)-(D-Arg)-Asn-Phe-NH₂

### Ac-(D-Arg)-(D-Arg)-Asn-Ile-NH₂

### Ac-(D-Arg)-(D-Arg)-Asn-Met-NH₂

### Ac-(D-Arg)-(D-Arg)-Asn-Val-NH₂

### Ac-(D-Arg)-(D-Arg)-Asn-Leu-NH₂

### Ac-Tyr-Asn-Arg-Arg-NH₂

### Ac-Tyr-Asn-Lys-Arg-NH₂

### Ac-Tyr-Asn-Arg-Lys-NH₂

### Ac-Tyr-Asn-Lys-Lys-NH 2

### Ac-(D-Tyr)-(D-Asn)-(D-Arg)-(D-Arg)-NH₂

### Ac-(D-Tyr)-(D-Asn)-(D-Lys)-(D-Arg)-NH₂

### Ac-(D-Tyr)-(D-Asn)-(D-Arg)-(D-Lys)-NH₂

### Ac-(D-Tyr)-(D-Asn)-(D-Lys)-(D-Lys)-NH₂

### Ac-Lys-Lys-Gln-Phe-NH₂

### Ac-Lys-Lys-Gln-Tyr-NH₂

### Ac-Lys-Lys-Gln-His-NH₂

### Ac-Lys-Lys-Gln-Trp-NH₂

### Ac-Lys-Lys-Gln-Val-NH₂

### Ac-Lys-Lys-Gln-Leu-NH₂

### Ac-Lys-Lys-GIn-Ile-NH₂

### Ac-Lys-Lys-Gln-Met-NH₂

### Ac-Arg-Arg-GIn-His-NH₂

### Ac-Arg-Arg-Gin-Trp-NH₂

### Ac-Arg-Arg-GIn-ILe-NH₂

### Ac-Arg-Arg-Gln-Met-NH₂

### Ac-Arg-Arg-Gln-Tyr-NH₂

### Ac-Arg-Arg-Gln-Val-NH₂

### Ac-Arg-Arg-Gln-Leu-NH₂

### Ac-Arg-Arg-Gln-Phe-NH₂

### Ac-Amp-Amp-Asn-Phe-NH₂

### Ac-Amp-Amp-Asn-Tyr-NH₂

### Ac-Amp-Amp-Asn-His-NH₂

### Ac-Amp-Amp-Asn-Trp-NH₂

### Ac-Amp-Amp-Asn-Val-NH₂

### Ac-Amp-Amp-Asn-Leu-NH₂

### Ac-Amp-Amp-Asn-Ile-NH₂

### Ac-Amp-Amp-Asn-Met-NH₂

### Ac-Arg-Lys-Asn-Phe-NH₂

### Ac-Arg-Lys-Asn-Tyr-NH₂

### Ac-Arg-Lys-Asn-His-NH₂

### Ac-Arg-Lys-Asn-Trp-NH₂

### Ac-Arg-Lys-Asn-Val-NH₂

### Ac-Arg-Lys-Asn-Leu-NH₂

### Ac-Arg-Lys-Asn-Ile-NH₂

### Ac-Arg-Lys-Asn-Met-NH₂

### Ac-Lys-Arg-Asn-Phe-NH₂

### Ac-Lys-Arg-Asn-Tyr-NH₂

### Ac-Lys-Arg-Asn-His-NH₂

### Ac-Lys-Arg-Asn-Trp-NH₂

### Ac-Lys-Arg-Asn-Val-NH₂

### Ac-Lys-Arg-Asn-Leu-NH₂

### Ac-Lys-Arg-Asn-Ile-NH₂

### Ac-Lys-Arg-Asn-Met-NH₂

Ac-Arg-Arg-Asn(CONH-(4-hydroxybenzyl))-NH₂
Ac-Arg-Arg-Lys(NH(4-hydroxybenzoyl))-NH₂

### Ac-Arg-Arg-Asn(CONH-(benzyl))-NH₂

### Ac-Arg-Arg-Lys(NH(benzoyl))-NH₂

Ac-Arg-Arg-Asn(CONH-(alfa-methylnaphthyl))-NH₂ Ac-Arg-Arg-Lys(NH(alfa-naphthoyl))-NH₂ Ac-Lys(4-hydroxybenzoyl)-Arg-Arg-NH₂

Ac-Lys-Lys-Asn(CONH-(4-hydroxybenzyl))-NH₂
Ac-Lys-Lys-Lys(NH (4-hydroxybenzoyl))-NH₂

### Ac-Lys-Lys-Asn(CONH-(benzyl))-NH₂

Ac-Lys-Lys-Lys(NH (benzoyl))-NH₂

### Ac-Lys-Lys-Asn(CONH-(alfa-methylnaphthyl))-NH₂

### Ac-Lys-Lys-Lys(NH(alfa-naphthoyl))-NH₂

Ac-Arg-Arg-Damba(NH (4-hydroxybenzoyl))-NH₂
Ac-Arg-Arg-Orn(NH(4-hydroxybenzoyl))-NH₂
Ac-Arg-Arg-Dab(NH(4-hydroxybenzoyl))-NH₂
Ac-Arg-Arg-Dapa(NH(4-hydroxybenzoyl))-NH₂
Ac-Arg-Arg-Daa(NH(4-hydroxybenzoyl))-NH₂
Ac-Arg-Arg-Amp(NH(4-hydroxybenzoyl))-NH₂
Ac-Arg-Arg-(4S-Amoa)(NH(4-hydroxybenzoyl))-NH₂
Ac-Arg-Arg-Phe(4-NH(4-hydroxybenzoyl))-NH₂
Ac-Arg-Arg-Ameg(NH(4-hydroxybenzoyl))-NH₂
Ac-Arg-Arg-pmGly(NH(4-hydroxybenzoyl))-NH₂
Ac-Arg-Arg-Ica(NH(4-hydroxybenzoyl))-NH₂
Ac-Arg-Arg-Apa(NH(4-hydroxybenzoyl))-NH₂
Ac-Lys-Lys-Damba(NH(4-hydroxybenzoyl))-NH₂
Ac-Lys-Lys-Orn(NH(4-hydroxybenzoyl))-NH₂
Ac-Lys-Lys-Dab(NH(4-hydroxybenzoyl))-NH₂
Ac-Lys-Lys-Dapa(NH(4-hydroxybenzoyl))-NH₂
Ac-Lys-Lys-Daa(NH(4-hydroxybenzoyl))-NH₂
Ac-Lys-Lys-Amp(NH(4-hydroxybenzoyl))-NH₂
Ac-Lys-Lys-(4S-Amoa)(NH(4-hydroxybenzoyl))-NH₂
Ac-Lys-Lys-Phe(4-NH (4-hydroxybenzoyl))-NH₂
Ac-Lys-Lys-Ameg(NH(4-hydroxybenzoyl))-NH₂
Ac-Lys-Lys-pmGly(NH(4-hydroxybenzoyl))-NH₂
Ac-Lys-Lys-Ica(NH(4-hydroxybenzoyl))-NH₂
Ac-Lys-Lys-Apa(NH(4-hydroxybenzoyl))-NH₂
Ac-Lys-Arg-Damba(NH(4-hydroxybenzoyl))-NH₂
Ac-Lys-Arg-Orn(NH(4-hydroxybenzoyl))-NH₂
Ac-Lys-Arg-Dab(NH(4-hydroxybenzoyl))-NH₂
Ac-Lys-Arg-Dapa(NH(4-hydroxybenzoyl))-NH₂
Ac-Lys-Arg-Daa(NH(4-hydroxybenzoyl))-NH₂
Ac-Lys-Arg-Amp(NH(4-hydroxybenzoyl))-NH₂
Ac-Lys-Arg-(4S-Amoa)(NH(4-hydroxybenzoyl))-NH₂
Ac-Lys-Arg-Phe(4-NH(4-hydroxybenzoyl))-NH₂
Ac-Lys-Arg-Ameg(NH(4-hydroxybenzoyl))-NH₂
Ac-Lys-Arg-pmGly(NH(4-hydroxybenzoyl))-NH₂
Ac-Lys-Arg-Ica(NH(4-hydroxybenzoyl))-NH₂
Ac-Lys-Arg-Apa(NH(4-hydroxybenzoyl))-NH₂
Ac-Arg-Lys-Damba(NH(4-hydroxybenzoyl))-NH₂
Ac-Arg-Lys-Orn(NH(4-hydroxybenzoyl))-NHhd 2
Ac-Arg-Lys-Dab(NH(4-hydroxybenzoyl))-NH₂
Ac-Arg-Lys-Dapa(NH(4-hydroxybenzoyl))-NH₂
Ac-Arg-Lys-Daa(NH(4-hydroxybenzoyl))-NH₂
Ac-Arg-Lys-Amp(NH(4-hydroxybenzoyl))-NH₂
Ac-Arg-Lys-(4S-Amoa)(NH (4-hydroxybenzoyl))-NH₂
Ac-Arg-Lys-Phe(4-NH(4-hydroxybenzoyl))-NH₂
Ac-Arg-Lys-Ameg (NH (4-hydroxybenzoyl))-NH₂
Ac-Arg-Lys-pmGly(NH(4-hydroxybenzoyl))-NH₂
Ac-Arg-Lys-Ica(NH(4-hydroxybenzoyl))-NH₂
Ac-Arg-Lys-Apa(NH(4-hydroxybenzoyl))-NH₂

### Ac-Lys-Lys-Asn-Tyr-Lys-Lys-Asn-Tyr-NH₂

### Ac-Arg-Arg-Asn-Tyr-Arg-Arg-Asn-Tyr-NH₂

### Ac-Arg-Lys-Asn-Tyr-Arg-Lys-Asn-Tyr-N1-1₂

### Ac-Arg-Lys-Asn-Tyr-Lys-Arg-Asn-Tyr-NH₂

### Ac-Arg-Arg-Asn-Tyr-Lys-Lys-Asn-Tyr-NN₂

### Ac-Lys-Lys-Asn-Tyr-Arg-Arg-Asn-Tyr-NH₂

More specific peptides within the scope of the invention are shown in Table 1 (below).

TABLE 1

| | MW | MW | |
|---|---|---|---|
| | monoisot. | monoisot. | |
| Compound | Calc. | Found | |
| # | (g/mol) | (g/mol) | Sequence |
| 2496 | 2384.16 | 2384.32 | Ac-Tyr-Asn-Arg-Arg-Gly- |
| | | | Gly-Gly-Ser-Ala-Val- |
| | | | Pro-Phe-Tyr-Ser-His- |
| | | | Ser-Tyr-Asn-Arg-Arg-NH2 |
| 2497 | 1237.65 | 1237.7 | Ac-Arg-Arg-Asn-Tyr-Arg- |
| | | | Arg-Asn-Tyr-NH2 |
| 2498 | 648.35 | 648.47 | Ac-Arg-Arg-Asn-Tyr-NH2 |
| 2499 | 648.35 | 648.41 | Ac-Tyr-Asn-Arg-Arg-NH2 |
| 2506 | 2213.1 | 2213.21 | Ac-Arg-Arg-Asn-Tyr-Ser- |
| | | | Ala-Val-Pro-Phe-Tyr- |
| | | | Ser-His-Ser-Arg-Arg- |
| | | | Asn-Tyr-NH2 |
| 2507 | 1548.78 | 1548.9 | Ac-Arg-Arg-Asn-Tyr-Ser- |
| | | | His-Ser-Arg-Arg-Asn- |
| | | | Tyr-NH2 |
| 2508 | 2073.04 | 2073.18 | Ac-Arg-Arg-Asn-Tyr-Gly- |
| | | | Gly-Gly-Ser-Ala-Val- |
| | | | Pro-Phe-Tyr-Arg-Arg- |
| | | | Asn-Tyr-NH2 |
| 2509 | 1408.72 | 1408.89 | Ac-Arg-Arg-Asn-Tyr-Gly- |
| | | | Gly-Gly-Arg-Arg-Asn- |
| | | | Tyr-NH2 |
| 2510 | 2240.11 | 2240.77 | Ac-Arg-Arg-Asn-Tyr-Gly- |
| | | | Gly-Ser-Ala-Val-Pro- |
| | | | Phe-Tyr-Ser-His-Arg- |
| | | | Arg-Asn-Tyr-NH2 |
| 2511 | 1651.88 | 1651.91 | Ac-Arg-Arg-Asn-Tyr-Ala- |
| | | | Val-Pro-Phe-Arg-Arg- |
| | | | Asn-Tyr-NH2 |
| 2512 | 1433.78 | 1433.9 | Ac-Arg-Arg-Asn-Tyr-Val- |
| | | | Pro-Arg-Arg-Asn-Tyr-NH2 |
| 2366 | 2384.16 | 2384.43 | Ac-Arg-Arg-Asn-Tyr-Gly- |
| | | | Gly-Gly-Ser-Ala-Val- |
| | | | Pro-Phe-Tyr-Ser-His- |
| | | | Ser-Arg-Arg-Asn-Tyr-NH2 |
| 2369 | 2600.52 | 2600.6 | Ac-Arg-Arg-Asn-Tyr-Ala- |
| | | | Ala-Leu-Ala-lys-Ala- |
| | | | Ala-Leu-Ala-Lys-Ala- |
| | | | Ala-Leu-Ala-Lys-Arg- |
| | | | Arg-Asn-Tyr-NH2 |
| 2370 | 1382.73 | 1382.88 | Ac-Arg-Arg-Asn-Tyr-(8- |
| | | | amino-3,6-dioxa- |
| | | | octanoicacid)-Arg-Arg- |
| | | | Asn-Tyr-NH2 |
| 2371 | 4119.98 | 4120.01 | Ac-Arg-Arg-Asn-Tyr-Gly- |
| | | | Gly-Gly-Ser-Ala-Val- |
| | | | Pro-Phe-Tyr-Ser-His- |
| | | | Ser-Arg-Arg-Asn-Tyr- |
| | | | Gly-Gly-Gly-Ser-Ala- |
| | | | Val-Pro-Phe-Tyr-Ser- |
| | | | His-Ser-Arg-Arg-Asn- |
| | | | Tyr-NH2 |
| 2372 | 5855.8 | 5855.94 | Ac-Arg-Arg-Asn-Tyr-Gly- |
| | | | Gly-Gly-Ser-Ala-Val- |
| | | | Pro-Phe-Tyr-Ser-His- |
| | | | Ser-Arg-Arg-Asn-Tyr- |
| | | | Gly-Gly-Gly-Ser-Ala- |
| | | | Val-Pro-Phe-Tyr-Ser- |
| | | | His-Ser-Arg-Arg-Asn- |
| | | | Tyr-Gly-Gly-Gly-Ser- |
| | | | Ala-Val-Pro-Phe-Tyr- |
| | | | Ser-His-Ser-Arg-Arg- |
| | | | Asn-Tyr-NH2 |
| 2517 | 560.27 | 560.3 | H-(2S,4R)Amp((2S,4R) |
| | | | Amp(Ac))-Asn-Tyr-NH2 |
| 2518 | 575.33 | 575.38 | Ac-Arg-Arg-Asn(CONH- |
| | | | BzI)-NH2 |
| 2519 | 619.36 | 619.41 | Ac-Arg-Arg-Lys(NH-4- |
| | | | hydroxybenzoyl)-NH2 |
| 2520 | 619.36 | 619.38 | Ac-Lys(NH-4-hydroxy- |
| | | | benzyol)Arg-Arg-NH2 |

Another example peptide according to the invention is compound 2624: Ac-Lys-Lys-Lys(4-hydroxybenzoyl)-NH₂.

In most of the example peptides listed above, R¹ is Ac and R² is NH₂. It will be understood that the R¹ and R² groups of the peptides listed above are not limited to the R¹ and R^{2 groups shown, and can be any R}¹ and R² groups within the scope of the invention. The R¹ and R² groups shown above may be the preferred R¹ and R² groups for the listed peptides.

More particular peptides according to the invention in one aspect facilitate and/or maintain or inhibit the intercellular communication mediated by gap junctions. The invention also relates to the preparation and use of pharmaceutical compositions for the treatment of pathologies associated with impaired intercellular gap junctional communication and methods for using these compositions.

### DEFINITIONS

Unless specified otherwise, the following definitions are provided for specific terms which are used in the following written description.

Throughout the description and claims the three-letter code for natural amino acids is used as well as generally accepted three letter codes for other α-amino acids, such as Sarcosin (Sar). Where the L or D form has not been specified, it is to be understood that the amino acid in question can be either the L or D form.

The term “halogen” refers to F, CI, Br, and I, where F and I are preferred.

The term “alkyl” refers to univalent groups derived from alkanes by removal of a hydrogen atom from any carbon atom: CₙH₂ₙ₊₁—. The groups derived by removal of a hydrogen atom from a terminal carbon atom of unbranched alkanes form a subclass of normal alkyl (n-alkyl) groups: H[CH₂]ₙ—. The groups RCH₂—, R₂CH— (R not equal to H), and R₃C— (R not equal to H) are primary, secondary and tertiary alkyl groups respectively. C(1-22)alkyl refers to any alkyl group having from 1 to 22 carbon atoms and includes C(1-6)alkyl, such as methyl, ethyl, propyl, iso-propyl, butyl, pentyl and hexyl and all possible isomers thereof.

By the phrase “lower alkyl” is meant a linear or branched alkyl having less than about 6 carbon atoms, preferably methyl, ethyl, propyl, or butyl.

The term “alkenyl” refers to a straight or branched or cyclic hydrocarbon group containing one or more carbon-carbon double bonds. C(2-22)alkenyl refers to any alkenyl group having from 2 to 22 carbon atoms and includes C(2-6)alkenyl, vinyl, allyl, 1-butenyl, etc.

The term “aralkyl” refers to aryl C(1-22)alkyl, and the term “aryl” throughout this specification means phenyl or naphthyl.

By aroyl is meant a group with the structure:

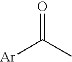

wherein Ar is aryl.

The phrase “hydrophobic group” includes an optionally substituted aromatic carbon ring, preferably a 6- to 12-membered aromatic carbon ring. The hydrophobic group can be optionally substituted as discussed below. Illustrative hydrophobic groups include benzyl, phenyl, and naphthyl.

The term “optionally substituted” as used herein means one or more hydrogen atoms (e.g., 1, 2, 3, 4, 5, or 6 hydrogen atoms) of the group can each be replaced with a substituent atom or group commonly used in pharmaceutical chemistry. Each substituent can be the same or different. Examples of suitable substituents include hydroxyl, primary amine (i.e., NH₂), secondary amine, tertiary amine, amide, carbamate, urea, hydrazide, halide, nitrile, nitro, sulfide, sulfoxide, sulfone, sulfonamide, thiol, carboxy, aldehyde, keto, carboxylic acid, ester, amide, imine, and imide, including thio derivatives thereof. Preferably, 1-3 optional substituents can be present, In embodiments in which a hydrophobic group is represented by a monocyclic aromatic carbon ring, preferably it is substituted in the 4 position, and preferably the substituent is hydroxyl.

By Glx(CONH-B) is meant a moiety having the following structure:

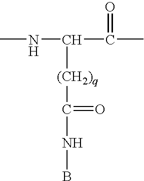

The term “Lysine mimetic” (LM) as used herein means a moiety having one of the following structures:

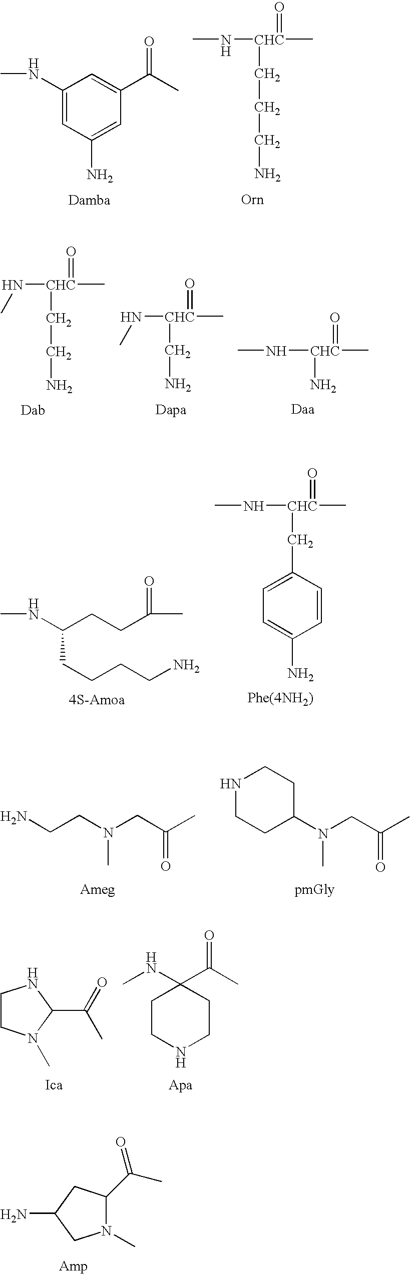

wherein Amp may be employed as an ex-amino acid (as shown above), or as a γ-amino acid by attaching to an adjacent amino acid or further lysine mimetic via the N atom of the NH₂ group shown above. An example of this is compound 2517:
H-(2S4R)Amp((2S4R)Amp(Ac))-Asn-Tyr-NH₂, which has the structure:

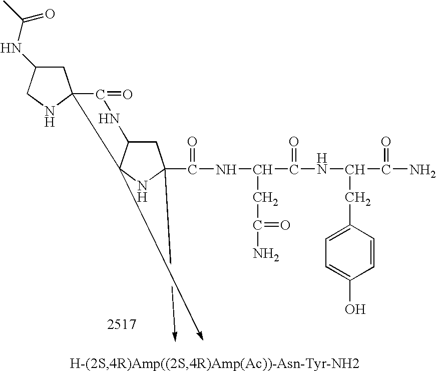

The second Amp (i.e. the Amp emboldened in H-(2S4R)Amp((2S4R)Amp(Ac))-Asn-Tyr-NH₂) is shown in parentheses because it is attached to the N atom of the NH₂ group of the side chain of the first Amp (i.e. the first Amp is employed as a γ-amino acid). The second Amp is acetylated, so the acetyl group is in parentheses after the second Amp.

The lysine mimetics in the peptides of the invention may be optionally substituted, wherein the substituent is preferably Ac.

Examples of how LM could be incorporated in the peptide sequences Z and Q are:

### For R1-Z-R2:

### R1-LM(NH₂)-A2-A3-A4-R2

### R1-LM(NH₂)-LM(NH₂)-A3-A4-R2

### R1-A1-LM(NH₂)-A3-A4-R2

### For R2-Z-L-Q-R1:

### R1-LM(NH₂)-A2-A3-A4-L-A5-A6-A7-A8-R2

### R1-LM(NH₂)-LM(NI-1₂)-A3-A4-L-A5-A6-A7-A8-R2

### R1-A1-LM(NH₂)-A3-A4-L-A5-A6-A7-A8-R2

### R1-A1-A2-LM(NH-B)-L-A5-A6-A7-A8-R2

### R1-LM(NH₂)-A2-A3-A4-L-LM(NH₂)-A6-A7-A8-R2

### R1-LM(NH₂)-LM(NH₂)-A3-A4-L-LM(NH₂)-LM(NH₂)-A7-A8-R2

### R1-A1-LM(NH₂)-A3-A4-L-A5-LM(NH₂)-A7-A8-R2

When the lysine mimetic is modified with B, the following moieties are meant:

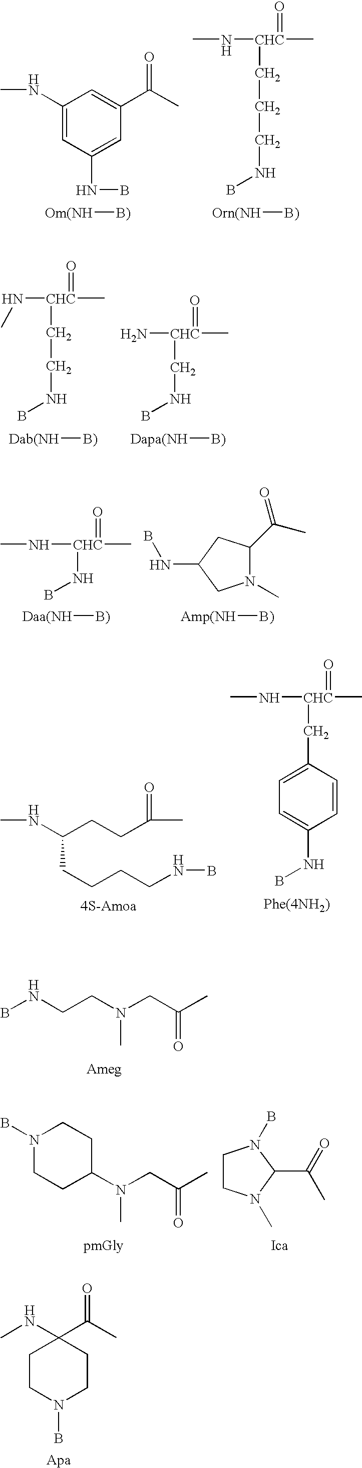

Examples of incorporation of LM(NH-B) into the peptides of the present invention are:

### For R1-Z-R2:

### R1-A1-A2-LM(NH-B)-R2

### R1-LM-A2-LM(NH-B)-R2

### R1-LM-LM-LM(NH-B)-R2

### R1-A1-LM-LM(NH-B)-R2

### For R2-Z-L-Q-R1:

### R1-A1-A2-LM(NH-B)-L-A5-A6-LM(NH-B)-R2

### R1-A1-A2-LM(NH-B)-L-LM(NH₂)-A6-LM(NH-B)-R2

### R1-A1-A2-LM(NH-B)-L-A5-LM(NH₂)-LM(NH-B)-R2

### R1-A1-A2-LM(NH-B)-L-LM(NH₂)-LM(NH₂)-LM(NH-B)-R2

### R1-LM(NH₂)-A2-LM(NH-B)-L-A5-A6-LM(NH-B)-R2

### R1-A1-LM(NH₂)-LM(NH-B)-L-LM(NH₂)-A6-LM(NH-B)-R2

### R1-LM(NH₂)-LM(NH₂)-LM(NH-B)-L-A5-LM(NH₂)-LM(NH-B)-R2

### R1-LM(NH₂)-LM(NH₂)-LM(NH-B)-L-LM(NH₂)-LM(NH₂)-LM(NH-B)-R2

### Etc.

Examples of retro analogues:

### R1-A4-A3-A2-A1-R1

### R1-A4-A3-LM(NH₂)-LM(NH₂)-R2

### R1-LM(NH-B)-A2-A1-R2

The carbon numbers used in the definitions herein (e.g., C₁₋₁₀ alkyl, C₂₋₁₀ alkenyl, C₂₋₁₀ alkynyl, C₆₋₂₀ aryl, etc.) refer to the carbon backbone and carbon branching, but do not include carbon atoms of substituents.

The terms “intercellular communication modulator”, “gap junction facilitator”, “compound that facilitates gap junction communication” and “gap junction opener”, etc., all refer to a compound that facilitates, or maintains, or normalizes (e.g. either by inhibiting of enhancing), GJIC, irrespective of the particular mechanism behind this action. More specifically, the term “gap junction opener” may refer to a substance which normalizes (i.e., increases) the exchange of molecules that are able to pass through gap junctions between extracellular and intracellular spaces and/or which can normalize increase GJIC.

The term “agonist” refers to a peptide that can interact with a tissue, cell or cell fraction which is the target of any given peptide, causing the same, or at least substantially the same physiological response.

The term “antagonist” refers to a peptide which inhibits or antagonizes one or more physiological responses observed in a tissue, cell or cell fraction after contacting the tissue, cell, or cell fraction with any given peptide.

As used herein, “normalize” refers to a change in a physiological response such that the response becomes insignificantly different from one observed in a normal patient. Thus, normalization may involve an increase or decrease in the response depending on the pathology involved.

Abbreviations used in the instant application are defined further below.

| | |
|---|---|
| Methyl (Me) | —CH₃ |
| Methoxy (MeO) | —OCH₃ |
| Ethyl (Et) | —CH₂CH₃ |
| Ethoxy (EtO) | —OCH₂CH₃ |
| n-Butyl (n-Bu) | —CH₂CH₂CH₂CH₃ |
| n-Butoxy (n-BuO) | —OCH₂CH₂CH₂CH₃ |
| n-Hexyl (n-Hex) | —CH₂CH₂CH₂CH₂CH₂CH₃ |
| n-Hexyloxy (n-HexO) | —OCH₂CH₂CH₂CH₂CH₂CH₃ |
| n-Octyl (n-Oct) | —CH₂CH₂CH₂CH₂CH₂CH₂CH₂CH₃ |
| n-Octyloxy (n-OctO) | —OCH₂CH₂CH₂CH₂CH₂CH₂CH₂CH₃ |
| t-Butyl (t-Bu) | 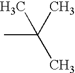 |
| t-Butoxy (t-BuO) | 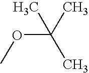 |
| cyclo-hexyl (c-Hex) | 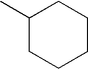 |
| cyclo-hexyloxy (c-HexO) | 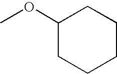 |
| Phenyl (Ph) | 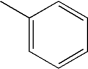 |
| Phenoxy (PhO) | 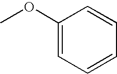 |
| Benzyl (Bzl) | 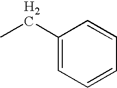 |
| Benzyloxy (BzlO) | 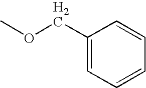 |

All references cited herein are incorporated by reference in their entireties herein.

Variations, modifications, and other implementations of what is described herein will occur to those of ordinary skill in the art without departing from the spirit and scope of the invention and the claims herein.

### Treatment Methods and Indications

In one aspect, the invention provides a method of administering to a patient having, or at risk of developing, a condition associated with impaired GJIC, a therapeutically effective amount of any of the peptides described above. Patients who may be treated using peptides according to the invention include, but are not limited to, animals, preferably mammals, e.g., rodents (including mice, rats, hamsters, and lagomorphs, such as rabbits), dogs, pigs, goats (generally any domestic animal), and primates. In one preferred aspect, a patient is a human being.

In another aspect the invention concerns the use of a peptide according to the invention for the manufacture of a medicament for the treatment of a pathological condition involving impaired gap junctional communication comprising administering to a patient a therapeutically effective amount of said peptide.

In a further aspect of the invention, there are provide peptides as described herein for us in methods of medical treatment. Preferably the method of medical treatment is treating a pathological condition involving impaired gap junctional communication.

Examples of conditions which can be treated include, but are not limited to, cardiovascular disease, inflammation of airway epithelium, disorders of alveolar tissue, bladder incontinence, impaired hearing due to diseases of the cochlea, endothelial lesions, diabetic retinopathy and diabetic neuropathy, ischemia of the central nervous system and spinal cord, dental tissue disorders including periodontal disease, kidney diseases, failures of bone marrow transplantation, wounds, erectile dysfunction, urinary bladder incontinence, neuropathic pain, subchronic and chronic inflammation, cancer and failures of bone marrow and stem cell transplantation, conditions which arise during transplantation of cells and tissues or during medical procedures such as surgery; as well as conditions caused by an excess of reactive oxygen species and/or free radicals and/or nitric oxide.

As discussed, it is an object of the invention to provide peptides that modulate gap junction intercellular communication (GJIC). Thus, many peptides in accord with the invention may include one or more of the following features: the ability to decrease cellular uncoupling, to normalize dispersion of action potential duration, and to normalize conduction velocity, the ability to control of the cellular quantity of gap junctions normalizing (up-regulating or down-regulating as needed) the expression of connexins; to normalize degradation of gap junctions (inhibit or enhance), to normalize cellular trafficking of connexins to the plasma membrane (increase or decrease); to facilitate assembly of connexins into functional gap junctions; to normalize opening of existing gap junctions, e.g., inducing or enhancing opening when they have been closed or gated by inhibitors (e.g., such as by mediating or enhancing hyperphosphorylation of the cytoplasmic carboxy terminal domain of one or more connexins (e.g., such as Cx43)) or closing these when they are aberrantly opened (e.g., as in Charcot-Marie-Tooth disease); and the like.

### Arrhythmia

In one preferred aspect, the invention provides a pharmacologically active antiarrhythmic peptide, and the use thereof, for treatment of arrhythmias and thrombotic complications arising during cardiovascular disorders, such as acute ischemic heart disease (e.g., stable angina pectoris, unstable angina pectoris, acute myocardial infarction), congestive heart failure (e.g., systolic, diastolic, high-output, low-output, right or left sided heart failure), congenital heart diseases, cor pulmonale, cardiomyopathies, myocarditis, hypertensive heart disease, during coronary revascularization, and the like.

In specific embodiments, an antiarrhythmic peptide according to the present invention is used to treat and/or prevent bradyarrhythmias (e.g., due to disease in sinus node, AV node, bundle of His, right or left bundle branch), and tachyarrhythmias associated with reentry (e.g., atrial premature complexes, AV junctional complexes, ventricular premature complexes, atrial fibrillation, atrial flutter, paroxymal supraventricular tachycardia, sinus node reentrant tachycardia, AV nodal reentrant tachycardia, and non-sustained ventricular tachycardia) either alone or in combination with other antiarrhythmic compounds, such as class I agents (e.g., lidocaine), class II agents (e.g., metoprolol or propranolol), class III agents (e.g., amiodarone or sotalol) or class IV agents (e.g., verapamil).

Additionally, or alternatively, peptides according to the invention are used to treat one or more of: a reentry arrhythmia; ventricular reentry (e.g., such as arises during acute myocardial infarction, chronic myocardial infarction, stable angina pectoris and unstable angina pectoris); infectious or autonomic cardiomyopathy; atrial fibrillation;repolarization alternans; monomorphic ventricular tachycardia; T-wave alternans; bradyarrhythmias; and generally, reduced contractility of cardiac tissue, thrombosis and the like.

### Osteoporosis

There is understanding that GJIC is important in bone formation. Additionally preferred peptides additionally, or alternatively, increase osteoblast activity in what is referred to herein as a “standard osteoblast activity assay” which measures either calcium wave formation and/or alkaline phosphatase activity of osteoblast cells in the presence of peptides. Preferably, such peptides increased calcium wave activity, manifested as an increase in the number of cells involved in a wave (as determined by measuring levels of intracellular Ca²⁺using a calcium sensitive fluorescent dye, such as fura-2 and counting the number of cells which fluoresce). Alkaline phosphatase activity also can be used to provide a measure of osteoblast activity using standard colorimetric assays.

In a further aspect, peptides according to the invention are used to prevent and/or treat osteoporosis or other pathologies affecting bone formation, growth or maintenance. Peptides which are able to normalize the attenuated GJIC between human osteoblast during hypoxia are particularly suitable for the treatment of bone diseases with impaired bone formation relative to bone resorption. Optimal peptides for use in such methods can be selected in assays for increased alkaline phosphatase (ALP) activity in osteoblasts, which provides a means to monitor cell viability and growth as a consequence of proper maintenance of GJIC. In one aspect, human osteoblasts are stimulated with different concentrations of peptides from 1×10⁻¹³ to 1×10⁻⁶, and compared to untreated controls. Under normal culture conditions, peptides preferably increase ALP activity. Even more preferably, the peptides stimulate ALP activity during hypoxic conditions at concentrations ranging from 10⁻¹¹ to 10⁻⁸ mol/l. The assay can thus be used to optimize peptide compositions for the treatment and/or prevention of bone diseases associated with poor vascularization, hypoxia and ischemia in bone tissue.

In another aspect, peptides according to the invention are used for the prevention and/or treatment of joint diseases that involves impaired cell-to-cell coupling. For example, the peptides can be used for the prevention and/or treatment of joint diseases that involve metabolic stress. These would include any form of arthritis associated with decreased vascularization or healing of fractured cartilage tissue.

### Cancer

In still another aspect, peptides according to the invention are used to treat cancer. Carcinogenesis is characterized by the progressive impairment of growth control mechanisms in which growth factors, oncogenes and tumor suppressor genes are involved. A general theme in carcinogenesis and tumorigenesis is the down regulation of the GJIC. Permeability of gap junctions in tumor cells using the dye-transfer assay is typically lower than GJIC in surrounding tissue. Further, the gating of gap junctions is known to be effected by tumor promoters, which decrease GJIC. Therefore, in one aspect, peptides according to the invention are used as medicaments for the treatment of cancer, alone, or in conjunction with traditional anti-cancer therapies.

### Wounds

In a further aspect, peptides according to the invention are used to treat wounds and, in particular, to accelerate wound healing. Wound healing involves the interactions of many cell types, and intercellular communication mediated by gap junctions is considered to play an important role in the coordination of cellular metabolism during the growth and development of cells involved in tissue repair and regeneration (K. M. Abdullah, et al. (1999) Endocrine, 10 35-41; M. Saitoh, et al. (1997) Carcinogenesis, 18: 1319-1328; J. A. Goliger, et al. (1995) Mol.Biol.Cell, 6 1491-1501). Peptides may be administered to the site of a wound by topical administration using carriers well known in the art (e.g., ointments, creams, etc.) or may administered systemically, e.g., for treating wounds of internal tissues, such as in the treatment of chronic gastric ulcer lesions.

Additional functions in which endothelial gap-junctional intercellular communication has been implicated are the migratory behavior of endothelial cells after injury, angiogenesis, endothelial growth and senescence, and the coordination of vasomotor responses (G. J. Christ, et al. (2000) Braz. J Med. Biol.Res., 33: 423-429). Therefore, in one aspect, a peptide according to the invention is used to enhance conducted vascular responses and to improve blood supply during conditions with increased metabolic demand (e.g., physical exercise, tachycardia), and during ischemia.

Gap junctions are also believed to provide the molecular link for coordinated long-range signaling among individual members of the glial compartment. Likewise, astrocytes are ideally suited for metabolic support of neurons since they are functionally polarized with one extremity touching the vascular bed and the other pole approximates neuronal parenchyma (R. Dermietzel (1998) Brain Res. Brain Res. Rev., 26: 176-183). Therefore, in one preferred embodiment, peptides according to the invention are administered to a patient in need to prevent ischemic damage in the brain by increasing the metabolic support between glia cells and neurons. Such patients may include patients with organic psychoses, which may present with signs such as depression, anxiety, learning and memory deficit, phobias, and hallucinations or patients who have suffered a traumatic brain injury. Preferably, such peptides are selected or formulated so as to be available to the central nervous system (i.e., provided with or conjugated with carriers which facilitate transport across the blood-brain barrier).

Peptides according to the invention may also be used to accelerate repair after nerve injury or during grafting of immature cells (progenitor cells) into brain tissue, e.g., such as in patients with neurotrauma, brain ischemia and chronic neurodegenerative diseases, such as Parkinson's disease or Huntington's disease (H. Aldskogius, et al. (1998) Prog. Neurobiol, 55: 1-26).

### Diabetes

Gap junction channels made of Cx43 functionally couples the glucose-sensitive cells of pancreatic islets and of a rat insulinoma cell line ^{[91]}. In contrast, cells of several cell lines secreting insulin abnormally do not express Cx43, have few gap junctions, and are poorly coupled. After correction of these defects by stable transfection of Cx43 cDNA, cells expressing modest levels of Cx43 and coupling, as observed in native beta-cells, show an expression of the insulin gene and an insulin content that is markedly elevated, compared with those observed in both wild-type (uncoupled) cells and in transfected cells overexpressing Cx43. These findings indicate that adequate coupling of Cx43 are required for proper insulin production and storage ^{[91]}. Moreover, in vivo stimulation of insulin release by glibenclamide is associated with increased expression of Cx43 and increased cell-to-cell coupling between neighbouring β-cells within the pancreatic islet ^{[92].}

These observations indicate an important role of gap junction coupling between pancreatic islet β-cells for the production and release of insulin. Thus, a still further purpose of the present invention is to provide a substance that increases the electrical conductance of gap junctions and, thus, improves glucose tolerance in subjects with non-insulin dependent diabetes mellitus.

In still another aspect, peptides according to the invention are used to treat diabetes. A still further purpose of the present invention is to provide a substance that increases the electrical conductance of gap junctions and, thus, improves glucose tolerance in subjects with non-insulin dependent diabetes mellitus.

In specific embodiments, a peptide according to the present invention may, due to the effect on the intercellular gap junction channels, be used to treat and/or prevent cataract (D. Mackay, et al. (1999) Am J Hum.Genet, 64 1357-1364) treat and/or prevent vascularization of the cornea in disease states with poor nutrition of the cornea and increase the healing of corneal lesions (S. G. Spanakis, et al. (1998) Invest Opthalmol.Vis.Sci., 39: 1320-1328) and/or prevent hypertension.

### Psoriasis

Psoriasis is a chronic skin disorder. Histologically, psoriasis is characterized by the hyperproliferation of the epidermis, elongated and prominent blood vessels and a thick perivascular lymphocytic infiltrate. Psoriasis is now considered an auto-immune disease. Although the disease occurs in all age groups, it primarily affects adults. It appears about equally in males and females. Psoriasis occurs when skin cells quickly rise from their origin below the surface of the skin and pile up on the surface before they have a chance to mature. Usually this movement (also called turnover) takes about a month, but in psoriasis it may occur in only a few days. In its typical form, psoriasis results in patches of thick, red (inflamed) skin covered with silvery scales. These patches, which are sometimes referred to as plaques, usually itch or feel sore. They most often occur on the elbows, knees, other parts of the legs, scalp, lower back, face, palms, and soles of the feet, but they can occur on skin anywhere on the body. Psoriasis is considered mild if it affects less than 5% of the surface of the body, moderate if 5-30% of the skin is involved, and severe if the disease affects more than 30% of the body surface.

Psoriasis usually involves the scalp and the extensor surfaces of the limbs especially the elbows and knees.

There are a number of psoriatic subtypes which describe the area of involvement, for example flexural psoriasis (where the skin lesions occur on flexor surfaces such as the groin), or the pattern of cutaneous change, for example psoriasis annularis (psoriasis with lesions occurring in ring shaped patches), or the type of cutaneous lesion, for example pustular psoriasis (where pustules predominate rather than papules, plaques or macules). Psoriatic arthritis, an erosive and usually asymmetrical oligoarthritis, may occur with this chronic recurrent papulosquamous skin disorder.

The symptoms of psoriasis may be mild, moderate or severe. There is currently no cure for psoriasis, but symptoms may be treated, depending on their severity.

Central to maintenance of healthy skin is a tight and highly ordered cell to cell adhesion network including adherens and tight junctions, desmosomes and gap junction intercellular communication channels (LAIRD, D.W. (2006). Life cycle of connexins in health and disease. Biochem J, 394, 527-43; PROCHNOW, N. & DERMIETZEL, R. (2008). Connexons and cell adhesion: a romantic phase. Histochem Cell Biol, 130, 71-7.). These intercellular junctions play crucial roles in epidermal proliferation, migration and differentiation and are all altered to differing degrees during psoriasis (CHUNG, E., COOK, P. W., PARKOS, C. A., PARK, Y. K., PITTELKOW, M. R. & COFFEY, R. J. (2005). Amphiregulin causes functional downregulation of adherens junctions in psoriasis. J Invest Dermatol, 124, 1134-40.; DJALILIAN, A. R., MCGAUGHEY, D., PATEL, S., SEO, E. Y., YANG, C., CHENG, J., TOMIC, M., SINHA, S., ISHIDA-YAMAMOTO, A. & SEGRE, J. A. (2006). Connexin 26 regulates epidermal barrier and wound remodeling and promotes psoriasiform response. J Clin Invest, 116, 1243-53.).

Expression of connexins, the constituent proteins of gap junctions, is spatially and temporally regulated within the epidermis with Cx43 in basal and spinous layers normally being replaced by Cx26 in the upper granular layers. In psoriasic plaques, however, Cx26 is greatly increased throughout the epidermis in a pattern similar to that in other hyperproliferative, stratified epithelia and appears to displace Cx43 (normally most abundant) in the spinous layers (LUCKE, T., CHOUDHRY, R., THOM, R., SELMER, I. S., BURDEN, A. D. & HODGINS, M. B. (1999). Upregulation of connexin 26 is a feature of keratinocyte differentiation in hyperproliferative epidermis, vaginal epithelium, and buccal epithelium. J Invest Dermatol, 112, 354-61.). In addition to the spatial redistribution of Connexins found in psoriasis there is also an associated down regulation of adheren junctions, in particular a marked reduction in E-cadherin is associated with the basal cell and upper spinous layers compared to normal epidermis (Chung et al 2005). Thus, the hyperproliferation and altered differentiation of psoriatic keratinocytes is associated with extensive remodelling of both gap-junctions and adherens junctions.

Thus, a still further purpose of the present invention is to provide a substance that keep the skin healthy by securing a tight and highly ordered cell to cell adhesion work including adherens and tight junctions, desmosomes and gap junction intercellular communication channels.

It should be obvious to those of skill in the art, that the peptides and pharmaceutical compositions according to the invention can be used to treat any condition or pathology associated with impaired (abnormal decreases or increases in) gap junctional communication. Preferably, one or more of the peptides or pharmaceutical compositions comprising the one or more peptides are administered to a patient in need thereof in a therapeutically effective amount. As used herein, “a therapeutically effective amount” is one which reduces symptoms of a given condition or pathology, and preferably which normalizes physiological responses in a patient with the condition or pathology. Reduction of symptoms or normalization of physiological responses can be determined using methods routine in the art and may vary with a given condition or pathology. In one aspect, a therapeutically effective amount of one or more peptides or pharmaceutical composition comprising the one or more peptides is an amount which restores a measurable physiological parameter to substantially the same value (preferably to within +30%, more preferably to within +20%, and still more preferably, to within 10% of the value) of the parameter in a patient without the DMF.

Particular assays useful for identifying and optionally quantifying the activity of preferred invention peptides are described below. The assays are non-limiting and are merely serving the purpose of illustrating a variety of assays in which the present peptides may be tested for their gap junction modulating abilities. It is to be understood that the assays are not mutually exclusive, i.e. a peptide according to the invention may show activity in one particular assay, but show a different, or no, activity in another particular assay. This may be a reflection of the diversity of the individual peptides and types of assays for testing said peptides.

### Pharmaceutical Compositions

The invention also concerns a pharmaceutical composition comprising one or more of any of the peptides described above, in combination with a pharmaceutically acceptable carrier and/or diluent. Formulations for administration may be prepared in a manner well-known to the person skilled in the art, e.g., as generally described in “Remington's Pharmaceutical Sciences”, 17. Ed. Alfonso R. Gennaro (Ed.), Mark Publishing Company, Easton, Pa., U.S.A., 1985 and more recent editions and in the monographs in the “Drugs and the Pharmaceutical Sciences” series, Marcel Dekker.

The pharmaceutical carrier or diluent employed may be a conventional solid or liquid carrier. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene and water.

Similarly, the carrier or diluent may include any sustained release material known in the art, such as glyceryl monostearate or glyceryl distearate, alone or mixed with a wax.

If a liquid carrier is used, the preparation may be in the form of a syrup or liquid suitable for oral ingestion.

It will be appreciated that the actual preferred amounts of active compounds used in a given therapy will vary according to e.g. the specific compound being utilized, the particular composition formulated, the mode of administration and characteristics of the subject, e.g. the species, sex, weight, general health and age of the subject. Optimal administration rates for a given protocol of administration can be readily ascertained by those skilled in the art using conventional dosage determination tests conducted with regard to the foregoing guidelines. Suitable dose ranges may include from about 1 mg/kg to about 100 mg/kg of body weight per day.

Therapeutic compounds of the invention may be suitably administered in a protonated and water-soluble form, e.g., as a pharmaceutically acceptable salt, typically an acid addition salt such as an inorganic acid addition salt, e.g., a hydrochloride, sulfate, or phosphate salt, or as an organic acid addition salt such as an acetate, maleate, fumarate, tartrate, or citrate salt. Pharmaceutically acceptable salts of therapeutic compounds of the invention also can include metal salts, particularly alkali metal salts such as a sodium salt or potassium salt; alkaline earth metal salts such as a magnesium or calcium salt; ammonium salts such an ammonium or tetramethyl ammonium salt; or an amino acid addition salts such as a lysine, glycine, or phenylalanine salt.

The compounds of the invention may also be administered topically to treat peripheral vascular diseases and as such may be formulated as a cream or ointment.

The present peptides may also be formulated in compositions such as sterile solutions or suspensions for parenteral administration such as intravenously, intramuscularly, subcutaneously, intranasally, intrarectally, intravaginally or intrapritoneally administration.

### EXAMPLES

The invention will now be further illustrated with reference to the following examples. It will be appreciated that what follows is by way of example only and that modifications to detail may be made while still falling within the scope of the invention.

### Example 1

### Peptide Synthesis

All peptides have been synthesized according to either solid phase or liquid phase procedures. However, more detailed descriptions of solid phase peptide syntheses are found in WO98/11125 and WO 2007/078990 hereby incorporated by reference in their entirety.

a. General Peptide Synthesis

### Apparatus and Synthetic Strategy

Peptides were synthesized batchwise in a polyethylene vessel equipped with a polypropylene filter for filtration using 9-fluorenylmethyloxycarbonyl (Fmoc) as N-α-amino protecting group and suitable common protection groups for side-chain functionalities.

### Solvents

Solvent DMF (N,N-dimethylformamide, Riedel de-Haen, Germany) was purified by passing through a column packed with a strong cation exchange resin (Lewatit S100 MB/H strong acid, Bayer AG Leverkusen, Germany) and analyzed for free amines prior to use by addition of 3,4-dihydro-3-hydroxy-4-oxo-1,2,3-benzotriazine (Dhbt-OH) giving rise to a yellow color (Dhbt-O⁻ anion) if free amines are present. Solvent DCM (dichloromethane, analytical grade, Riedel de-Häen, Germany) was used directly without purification. Acetonitril (HPLC-grade, Lab-Scan, Dublin Ireland) was used directly without purification.

### Amino Acids

Fmoc-protected amino acids were purchased from Advanced ChemTech (ACT) in suitable side-chain protected forms. Otherwise protected amino acids (Boc-Asp(OFm)-OH, Boc-D-Asp(OFm)-OH, Fmoc-Lys(Aloc)-OH, Fmoc-D-Lys(Aloc)-OH, Boc-Lys(Fmoc)-OH Boc-D-Lys(Fmoc)-OH, Boc-Orn(Fmoc)-OH from Bachem (Switzerland); Fmoc-Lys(ivDde)-OH, Fmoc-Sar-OH from NovaBiochem (Switzerland).

Benzoic acid and Benzyl Amine Derivatives

Benzoic acid and Benzyl amine derivatives were purchased from Aldrich and used without further purification.

### Coupling Reagents

Coupling reagent diisopropylcarbodiimide (DIC) was purchased from (Riedel de-Häen, Germany), PyBop from Advanced ChemTech.

### Linkers

(4-hydroxymethylphenoxy)acetic acid (HMPA), was purchased from Novabiochem, Switzerland; and was coupled to the resin as a preformed 1-hydroxybenzotriazole (HOBt) ester generated by means of DIC.

### Solid Supports

Peptides synthesized according to the Fmoc-strategy on TentaGel S resins 0.22-0.31 mmol/g (TentaGel-S-NH₂; TentaGel S-Ram, Rapp polymere, Germany).

### Catalysts and Other Reagents

Diisopropylethylamine (DIEA) was purchased from Aldrich, Germany, and ethylenediamine from Fluka, piperidine and pyridine from Riedel-de Häen, Frankfurt, Germany. 4-(N,N-dimethylamino)pyridine (DMAP) was purchased from Fluka, Switzerland and used as a catalyst in coupling reactions involving symmetrical anhydrides. Ethandithiol was purchased from Riedel-de Häen, Frankfurt, Germany. 3,4-dihydro-3-hydroxy-4-oxo-1,2,3-benzotriazine (Dhbt-OH), 1-hydroxybenzotriazole (HOBt) (HOAt) were obtained from Fluka, Switzerland.

### Coupling Procedures

The first amino acid can be coupled as a symmetrical anhydride in DMF generated from the appropriate n-α-protected amino acid and the subsequent amino acids can be coupled as in situ generated HOBt or HOAt esters made from appropriate n-α-protected amino acids and HOBt or HOAt by means of DIC in DMF. The acylations were checked by the ninhydrin test performed at 80 oc in order to prevent Fmoc deprotection during the test (B. D. Larsen, A. Holm, Int.J Pept.Protein Res. 1994, 43 1-9).

Deprotection of the N-α-amino Protecting Group (Fmoc)

Deprotection of the Fmoc group was performed by treatment with 20% piperidine in DMF (1×5 and 1×10 min.), followed by wash with DMF (5×15 ml, 5 min. each) until no yellow color could be detected after addition of Dhbt-OH to the drained DMF.

### Deprotection of Allyl/Aloc

A solution of 3 eq. Pd(PPh₃)₄ dissolved in 15-20 ml CHCl₃, AcOH, NMM (37:2:1) was added to the peptide resin. The treatment was continued for three hours at room temperature accompanied by bubbling a stream of N₂ through the mixture.

### Coupling Of Hobt-Esters

3 eq. N-α-amino protected amino acid was dissolved in DMF together with 3 eq. HOBt and 3 eq. DIC and then added to the resin.

### Preformed Symmetrical Anhydride

Six eq. N-α-amino protected amino acid was dissolved in DCM and cooled to 0° C. DIC (3 eq.) was added and the reaction continued for 10 minutes. The solvent was removed in vacuo and the remnants dissolved in DMF. The solution was immediately added to the resin followed by 0.1 eq. of DMAP.

### Cleavage Of Peptide From Resin With Acid

Peptides were cleaved from the resins by treatment with 95% trifluoroacetic acid (TFA, Riedel-de Häen, Frankfurt, Germany)-water v/v or with 95% TFA and 5% ethandithiol v/v at r.t. for 2 hours. The filtered resins were washed with 95% TFA-water and filtrates and washings evaporated under reduced pressure. The residue was washed with ether and freeze-dried from acetic acid-water. The crude freeze-dried product was analyzed by high-performance liquid chromatography (HPLC) and identified by electrospray ionisation mass spectrometry (ESMS).

### Batchwise Peptide Synthesis on TentaGel Resin (PEG-PS)

TentaGel resin (1g, 0.22-0.31 mmol/g) was placed in a polyethylene vessel equipped with a polypropylene filter for filtration. The resin was swelled in DMF (15 ml), and treated with 20% piperidine in DMF to secure the presence of non-protonated amino groups on the resin. The resin was drained and washed with DMF until no yellow color could be detected after addition of Dhbt-OH to the drained DMF. HMPA (3 eq.) was coupled as a preformed HOBt-ester as described above and the coupling was continued for 24 h. The resin was drained and washed with DMF (5×5 ml, 5 min each) and the acylation checked by the ninhydrin test. The first amino acid was coupled as a preformed symmetrical anhydride as described above. The following amino acids according to the sequence were coupled as preformed Fmoc-protected HOBt esters (3 eq.) as described above. The couplings were continued for 2 h, unless otherwise specified. The resin was drained and washed with DMF (5×15 ml, 5 min each) in order to remove excess reagent. All acylations were checked by the ninhydrin test performed at 80° C. After completed synthesis the peptide-resin was washed with DMF (3×15 ml, 5 min each), DCM (3×15 ml, 1 min each) and finally diethyl ether (3×15 ml, 1 min each) and dried in vacuo

Preparative HPLC conditions.

Preparative chromatography was carried out using a VISION Workstation (PerSeptive Biosystem) equipped with AFC2000 automatic fraction collector/autosampler. VISION-3 software was used for instrument control and data acquisition.

### Column

Kromasil (EKA Chemicals) KR100-10-C8 100 Å, C-8, 10□; CER 2230, 250×50.8 mm or a VYDAC 218TP101550, 300 Å, C-18, 10-15 □, 250×50 mm. The buffer system used included A: 0,1% TFA in MQV; B: 0,085% TFA, 10% MQV, 90% MeCN. Flow rates were 35-40 ml/min and the column temperature was 25° C. UV detection was performed at 215 nm and 280 nm. Suitable gradients were optimized for individual peptides.

### Analytical HPLC Conditions

Gradient HPLC analysis was done using a Hewlett Packard HP 1100 HPLC system consisting of a HP 1100 Quaternary Pump, a HP 1100 Autosampler a HP 1100 Column Thermostat and HP 1100 Multiple Wavelength Detector. Hewlett Packard Chemstation for LC software (rev. A.06.01) was used for instrument control and data acquisition.

For analytical HPLC, different columns were used as appropriate, such as VYDAC 238TP5415, C-18, 5 μm, 300 Å, or a Jupiter, Phenomenex 00E-4053-E0; 5 □m C-18, 300 Å150×4,6 mm and others. The buffer system included A: 0,1% TFA in MQV; B: 0,085% TFA, 10% MQV, 90% MeCN. Flow rates were 1 ml/min. The preferred column temperature was 40° C. UV detection was performed at 215 nm. As above, suitable gradients were optimized for the individual peptides.

### Mass Spectroscopy

The peptides were dissolved in super gradient methanol (Labscan, Dublin, Ireland), Milli-Q water (Millipore, Bedford, Mass.) and formic acid (Merck, Damstadt, Germany) (50:50:0.1 v/v/v) to give concentrations between 1 and 10 μg/ml. The peptide solutions (20 μl) were analysed in positive polarity mode by ESI-TOF-MS using a LCT mass spectrometer (Micromass, Manchester, UK) accuracy of +/−0.1 m/z.

### General Synthetic Procedure

In all syntheses, dry TentaGel-S-NH₂ (0.23 mmol/g, 1g) was placed in a polyethylene vessel equipped with a polypropylene filter for filtration and treated as described under “batchwise peptide synthesis on TentaGel resin”. Lysine and analogs thereof (e.g. Ornithin, 2,4-Diaminobutanoic acid, 1,3-diaminopropanoic acid etc.) when situated C-terminally were coupled as the N-□ Fmoc protected derivatives with either ivDde or Aloc protection of the side chain functionality (e.g. Fmoc-Lys(Aloc)—OH). Lysine and analogs thereof when situated N-terminally were coupled as the N-□ Boc protected derivatives with Fmoc protection of the side chain functionality (e.g. Boc-Lys(Fmoc)-OH). Aspartic acid, Glutamic acid and analogs thereof when situated C-terminally were coupled as N-□ Fmoc protected derivatives with Allylic protection of the side chain functionality (e.g. Fmoc-Asp(Oallyl)-OH). Aspartic acid, Glutamic acid and analogs thereof when situated N-terminally were coupled as N-□ Boc protected derivatives with Fm protection of the side chain functionality (e.g. Boc-Asp(OFm)-OH). Other amino acids than the above mentioned when situated C-terminally were coupled as N-□ Fmoc protected derivatives with suitable protection of the side chain functionalities or when situated N-terminally as N-□ Boc protected derivatives with suitable protection of the side chain functionalities.

In all cases the dipeptide was assembled followed by deprotection of the side chain protecting group of the Lysine, Aspartic- or Glutamic acid or analogs thereof.

In case of Lysine or analogs thereof, the hydrophobic group functionalised as a carboxylic acid was coupled as an in situ generated HOBt ester by means of DIC in THF.

In case of Aspartic- and Glutamic acid or analogs thereof, the hydrophobic group functionalised as an amine was coupled to the pre generated HOBt ester of the side chain carboxylic acid by means of DIC in DMF catalyzed by triethylamine.

All couplings were continued for at least 2 hours. The acylations were checked by the ninhydrin test performed at 80° C. as earlier described. After completed synthesis the peptide-resin was washed with DMF (3×15 ml, 1 min each), DCM (3×15 ml, 1 min each), diethyl ether (3×15 ml, 1 min each) and dried in vacuo. The peptide was then cleaved from the resin as described above and freeze-dried.

After purification using preparative HPLC as described above, the peptide product was collected and the identity of the peptide was confirmed by ES-MS .

The above procedure was used for the synthesis of all peptides exemplified further below and the peptides shown in Table 1 of the specification.

### Example 2

### Production of Recombinant Protein

Recombinant Cx43CT was produced as described in Duffy et al., “pH-Dependent Intramolecular Binding and Structure Involving Cx43 Cytoplasmic Domains,” J. Biol. Chem.

277(39):36706-36714 (2002), which is hereby incorporated by reference in its entirety. Briefly, cDNA derived from rat Cx43 was inserted into pGEX-6P-2 (Amersham) and expressed in E. coli (BL-21). The resultant GST-fusion protein was cleaved from the GST PreScission Protease® (Amersham Biosciences). The recombinant product after cleavage contained the sequence 255-382 of rCx43 preceded by four additional amino acids (GPLG). Protein concentration was measured using the Bio-Rad DC Protein Assay. Protein purity was assessed by SDS-PAGE.

### Example 3

### Surface Plasmon Resonance (SPR)

SPR is a spectroscopic method to determine binding amplitude and kinetics in real time (Salamon et al., “Surface Plasmon Resonance Spectroscopy as a Tool for Investigating the Biochemical and Biophysical Properties of Membrane Protein Systems. II: Applications to Biological Systems,” Biochim. Biophys. Acta 1331(2):131-152 (1997); Duffy et al., “Functional Demonstration of Connexin-protein Binding Using Surface Plasmon Resonance,” Cell Adhes. Commun. 8(4-6):225-229 (2001); Lang et al., “Surface Plasmon Resonance as a Method to Study the Kinetics and Amplitude of Protein-protein Binding,” in PRACTICAL METHODS IN CARDIOVASCULAR RESEARCH 936-947 (Stefan Dhein, Friedrich Wilhelm Mohr & Mario Delmar eds., 2005), which are hereby incorporated by reference in their entirety). Recombinant Cx43CT was covalently bound to a carboxylmethyl dextran matrix (Salamon et al., “Surface Plasmon Resonance Spectroscopy as a Tool for Investigating the Biochemical and Biophysical Properties of Membrane Protein Systems. II: Applications to Biological Systems,” Biochim. Biophys. Acta 1331(2):131-152 (1997), which is hereby incorporated by reference in its entirety). Specific peptides were presented and, when feasible, dissociation constants (K_{D}) were calculated from the time course of binding and unbinding of the ligand, using a 1:1 (Langmuir) association and dissociation kinetic model (Biacore software package). In both phases (association and dissociation), the first 5-8 seconds of recording were not included in the fit, to avoid artifacts resulting from peptide distribution within the flow cells (Lang et al., “Surface Plasmon Resonance as a Method to Study the Kinetics and Amplitude of Protein-protein Binding,” in PRACTICAL METHODS IN CARDIOVASCULAR RESEARCH 936-947 (Stefan Dhein, Friedrich Wilhelm Mohr & Mario Delmar eds., 2005), which is hereby incorporated by reference in its entirety).

### Example 3a

### Peptide Binding to Cx43 Determined Using SPR

The peptides shown in table 2 and 3 were tested for binding to the carboxyl terminal domain (CT) of Cx43 using Surface Plasmon Resonance (“SPR”). SPR is a method that was used to identify the original RXPE sequence (Shibayama et al., “Identification of a Novel Peptide that Interferes with the Chemical Regulation of Connexin-43,” Circ. Res. 98:1365-72 (2006)). The concentration of the peptide, relative to the amplitude of the response in arbitrary units, is shown in Table 2. The amplitude of the response is also a function of the mass of the ligate, and the normalized data according to the individual peptide Mw's are shown in table 3. The data show that peptides as small as four amino acids in length are able to bind to the carboxyl terminal domain (CT) of Cx43.

TABLE 2
SPR respons

| Cmpd # | Sequence | 1 mM | 500 uM | 250 uM | 100 uM | 25 uM |
|---|---|---|---|---|---|---|
| 2496 | Ac-Tyr-Asn-Arg-Arg-Gly-Gly-Gly-Ser- | 1402 | 738.6 | 358.1 | 155.9 | 67.7 |
| | Ala-Val-Pro-Phe-Tyr-Ser-His-Ser-Tyr- | | | | | |
| | Asn-Arg-Arg-NH2 | | | | | |
| 2497 | Ac-Arg-Arg-Asn-Tyr-Arg-Arg-Asn-Tyr- | 565.2 | 327.5 | 201.5 | 84.2 | 30.2 |
| | NH2 | | | | | |
| 2498 | Ac-Arg-Arg-Asn-Tyr-NH2 | 91 | 50.9 | n.d. | n.d. | n.d. |
| 2499 | Ac-Tyr-Asn-Arg-Arg-NH2 | 69.4 | n.d. | n.d. | n.d. | n.d. |
| 2506 | Ac-Arg-Arg-Asn-Tyr-Ser-Ala-Val-Pro- | 1172.8 | 561.8 | 327 | 117.2 | 51.7 |
| | Phe-Tyr-Ser-His-Ser-Arg-Arg-Asn-Tyr- | | | | | |
| | NH2 | | | | | |
| 2507 | Ac-Arg-Arg-Asn-Tyr-Ser-His-Ser-Arg- | 703.2 | 438.5 | 254.2 | 113.5 | 42.3 |
| | Arg-Asn-Tyr -NH2 | | | | | |
| 2508 | Ac-Arg-Arg-Asn-Tyr-Gly-Gly-Gly-Ser- | 1024.5 | 546.8 | 284.6 | 121.9 | 31.1 |
| | Ala-Val-Pro-Phe-Tyr-Arg-Arg-Asn-Tyr- | | | | | |
| | NH2 | | | | | |
| 2509 | Ac-Arg-Arg-Asn-Tyr-Gly-Gly-Gly-Arg- | 478.9 | 280.8 | 148.5 | 62.5 | 21.5 |
| | Arg-Asn-Tyr-NH2 | | | | | |
| 2510 | Ac-Arg-Arg-Asn-Tyr-Gly-Gly-Ser-Ala- | 1188 | 643.3 | 331.5 | 176.2 | 40.3 |
| | Val-Pro-Phe-Tyr-Ser-His-Arg-Arg-Asn- | | | | | |
| | Tyr-NH2 | | | | | |
| 2511 | Ac-Arg-Arg-Asn-Tyr-Ala-Val-Pro-Phe- | 691.5 | 389.6 | 214.2 | 90.5 | 16.2 |
| | Arg-Arg-Asn-Tyr-NH2 | | | | | |
| 2512 | Ac-Arg-Arg-Asn-Tyr-Val-Pro-Arg-Arg- | 394.7 | 227.3 | 117.2 | 50.7 | 14.8 |
| | Asn-Tyr-NH2 | | | | | |

TABLE 3
Suface Plasma ReSonance data (SPR) normalised on Mw

| Cmpd | Mw | | | | | | |
|---|---|---|---|---|---|---|---|
| # | monoisotop | Sequence | 1 mM | 500 uM | 250 uM | 100 uM | 25 uM |
| 2496 | 2384.16 | Ac-Tyr-Asn-Arg-Arg-Gly-Gly- | 58.8 | 31.0 | 15.0 | 6.5 | 2.8 |
| | | Gly-Ser-Ala-Val-Pro-Phe-Tyr- | | | | | |
| | | Ser-His-Ser-Tyr-Asn-Arg-Arg- | | | | | |
| | | NH2 | | | | | |
| 2497 | 1237.65 | Ac-Arg-Arg-Asn-Tyr-Arg-Arg- | 45.7 | 26.5 | 16.3 | 6.8 | 2.4 |
| | | Asn-Tyr-NH2 | | | | | |
| 2498 | 648.35 | Ac-Arg-Arg-Asn-Tyr-NH2 | 14.0 | 7.9 | | | |
| 2499 | 648.35 | Ac- Tyr-Asn-Ars-Arg-NH2 | 10.7 | | | | |
| 2506 | 2213.1 | Ac-Arg-Arg-Asn-Tyr-Ser-Ala- | 53.0 | 25.4 | 14.8 | 5.3 | 2.3 |
| | | Val-Pro-Phe-Tyr-Ser-His-Ser- | | | | | |
| | | Arg-Arg-Asn-Tyr-NH2 | | | | | |
| 2507 | 1548.78 | Ac- Arg-Arg-Asn-Tyr-Ser-His- | 45.4 | 28.3 | 16.4 | 7.3 | 2.7 |
| | | Ser-Arg-Arg-Asn-Tyr-NH2 | | | | | |
| 2508 | 2073.04 | Ac-Arg-Arg-Asn-Tyr-Gly-Gly- | 49.4 | 26.4 | 13.7 | 5.9 | 1.5 |
| | | Gly-Ser-Ala-Val-Pro-Phe-Tyr- | | | | | |
| | | Arg-Arg-Asn-Tyr-NH2 | | | | | |
| 2509 | 1408.72 | Ac-Arg-Arg-Asn-Tyr-Gly-Gly- | 34.0 | 19.9 | 10.5 | 4.4 | 1.5 |
| | | Gly-Arg-Arg-Asn-Tyr-NH2 | | | | | |
| 2510 | 2240.11 | Ac-Arg-Arg-Asn-Tyr-Gly-Gly- | 53.0 | 28.7 | 14.8 | 7.9 | 1.8 |
| | | Ser-Ala-Val-Pro-Phe-Tyr-Ser- | | | | | |
| | | His-Arg-Arg-Asn-Tyr-NH2 | | | | | |
| 2511 | 1651.88 | Ac-Arg-Arg-Asn-Tyr-Ala-Val- | 41.9 | 23.6 | 13.0 | 5.5 | 1.0 |
| | | Pro-Phe-Arg-Arg-Asn-Tyr-NH2 | | | | | |
| 2512 | 1433.78 | Ac-Arg-Arg-Asn-Tyr-Val-Pro- | 27.5 | 15.9 | 8.2 | 3.5 | 1.0 |
| | | Arg-Arg-Asn-Tyr-NH2 | | | | | |

Normalised on Mw means that the observed SPR response has been dived by the individual Mw for the peptides:
value = (SPRrespons*100)/Mw_{cmpd#}

### Example 4

### Nuclear Magnetic Resonance (NMR)

All NMR data may be acquired on a Varian INOVA 600-MHz NMR spectrometer using a cryoprobe (MacUra & Ernst, “Elucidation of Cross Relaxation in Liquids by Two-dimensional NMR Spectroscopy,” Mol. Phys. 41:95-117 (1980), which is hereby incorporated by reference in its entirety); the sample temperature is maintained at 7° C. Gradient-enhanced two-dimensional ¹H-¹⁵N HSQC experiments (Kay et al., “Pure Absorption Gradient Enhanced Heteronuclear Single Quantum Correlation Spectroscopy with Improved Sensitivity,” J. Am. Chem. Soc. 114:10663-10665 (1992), which is hereby incorporated by reference in its entirety) are used to observe all backbone amide resonances in ¹⁵N-labeled Cx43CT. Data are acquired with 1024 complex points in t₂ and 128 complex points in t₁. Sweep widths are 10,000 Hz in the proton dimension and 2,500 Hz in the nitrogen dimension. The concentration of peptide to Cx43CT was approximately 2.4 mM to 0.8 mM, respectively (3:1 ratio). All NMR data are processed using NMRPipe (Delaglio et al., “NMRPipe: A Multidimensional Spectral Processing System Based on UNIX Pipes,” J. Biomol. NMR 6(3):277-293 (1995), which is hereby incorporated by reference in its entirety) and analyzed using NMRView (Sorgen, “How to Solve a Protein Structure by Nuclear Magnetic Resonance—The Connexin43 Carboxyl Terminal Domain,” in PRACTICAL METHODS IN CARDIOVASCULAR RESEARCH 948-958 (Stefan Dhein, Friedrich Wilhelm Mohr & Mario Delmar eds., 2005), which is hereby incorporated by reference in its entirety).

### Example 5

### Electrophysiological Analysis

Experiments were conducted on N2a (Neuroblastoma) cells. Cx43 was expressed either in a lac-switch stable system (induced by 0.1-1.0 mM of IPTG (Zhong et al., “LacSwitch II Regulation of Connexin43 cDNA Expression Enables Gap-junction Single-channel Analysis,” Biotechniques 34(5):1034-1034, 1041-1044, 1046 (2003), which is hereby incorporated by reference in its entirety)) or transiently using an IRES plasmid coding for eGFP (Seki et al., “Modifications in the Biophysical Properties of Connexin43 Channels by a Peptide of the Cytoplasmic Loop Region,” Circ. Res. 95(4):e22-e28 (2004); Seki et al., “Loss of Electrical Communication, but Not Plaque Formation, After Mutations in the Cytoplasmic Loop of Connexin43,” Heart Rhythm. 1(2):227-233 (2004), which are hereby incorporated by reference in their entirety). M257 (a mutant of Cx43 truncated at amino acid 257 (Morley et al., “Intramolecular Interactions Mediate pH Regulation of Connexin43 Channels,” Biophys. J. 70(3):1294-1302 (1996), which is hereby incorporated by reference in its entirety)) was transiently expressed in N2a cells also using an eGFP-containing IRES plasmid. Cells were placed on the stage of an inverted microscope equipped for epifluorescence (Nikon Diaphoto200, Filter: 520 to 560 nm). Junctional current (I) was recorded from eGFP-positive cell pairs in a dual-whole-cell voltage clamp configuration (holding potentials: −40 mV; transjuctional voltage, Vⱼ, +60 MV; step duration, 10-30 seconds). Patch pipettes were filled with a cesium-containing solution (Seki et al., “Modifications in the Biophysical Properties of Connexin43 Channels by a Peptide of the Cytoplasmic Loop Region,” Circ. Res. 95(4):e22-e28 (2004); Seki et al., “Loss of Electrical Communication, but Not Plaque Formation, After Mutations in the Cytoplasmic Loop of Connexin43,” Heart Rhythm. 1(2):227-233 (2004), which are hereby incorporated by reference in their entirety). For the low pH experiments, HEPES was replaced by MES (10 mM). Pipette resistance was 4.0-6.0MΩ. Synthetic peptides were diluted in the pipette solution to a final concentration of 0.1 mM. During recording, cells were kept at room temperature in a cesium-containing solution (Seki et al., “Modifications in the Biophysical Properties of Connexin43 Channels by a Peptide of the Cytoplasmic Loop Region,” Circ. Res. 95(4):e22-e28 (2004); Seki et al., “Loss of Electrical Communication, but Not Plaque Formation, After Mutations in the Cytoplasmic Loop of Connexin43,” Heart Rhythm. 1(2):227-233 (2004), which are hereby incorporated by reference in their entirety). For some experiments, octanol (2.0 mM) was superfused during recording. Data acquisition and recording, and criteria for single channel detection were as reported in Seki et al., “Modifications in the Biophysical Properties of Connexin43 Channels by a Peptide of the Cytoplasmic Loop Region,” Circ. Res. 95(4):e22-e28 (2004) and Seki et al., “Loss of Electrical Communication, but Not Plaque Formation, After Mutations in the Cytoplasmic Loop of Connexin43,” Heart Rhythm. 1(2):227-233 (2004), which are hereby incorporated by reference in their entirety.

### Example 6

### Quantitative analysis of Cx43CT-compound interactions

### Specific Experiments

1) Determination of Cx43CT-compound dissociation constant. The binding kinetics of each compound to Cx43CT is assessed by SPR. Various concentrations of ligate are used and the rate of association and dissociation are fitted with a Langmuir 1:1 kinetic model (Biacore). This model assumes first-rate order kinetics of binding. Proper fitting yields an estimation of K_{D}. It is, however, possible that the interaction of the compound to the Cx43CT departs from a uni-uni model, in which case K_{D} values are not generated. In that case the experiments of cross-linking and NMR proposed below are relied on.

2) Concentration-dependence of binding using cross-linking reagents. This system allows us to establish a quantitative parameter for compound-Cx43Ct association even if the binding kinetics are not suitable for K_{D} determinations. Cross-linking is tested for a constant concentration of Cx43CT and sequential dilutions of the compound. It is expected to see that the decrease in the concentration of the compound will bring about a progressive increase in the density of the monomeric and dimeric Cx43CT band at the expense of the bands of lower mobility (i.e., those corresponding to compound-Cx43CT complexes). The ratio of bound versus unbound densities is plotted as a function of compound concentration. A sigmoidal concentration-dependence relation is expected. The peptide concentration corresponding to half-maximum binding is defined as the apparent EC50.

3) Identification of compound-induced Cx43CT resonance shifts by nuclear magnetic resonance (NMR). Previous studies have identified those amino acids in Cx43CT whose position in space is affected by RXP4 and by RXPE. The resonance map for Cx43CT is repeated with candidate compounds. It is our hypothesis that peptides with similar effects on function will also bind to the same amino acid regions.

### Example 7

### Functional Assessment by Dual Patch Clamp

Compounds were tested for their ability to interfere with octanol-induced uncoupling. Compounds are tested for their ability to interfere with acidification induces uncoupling.

### Specific Experiments

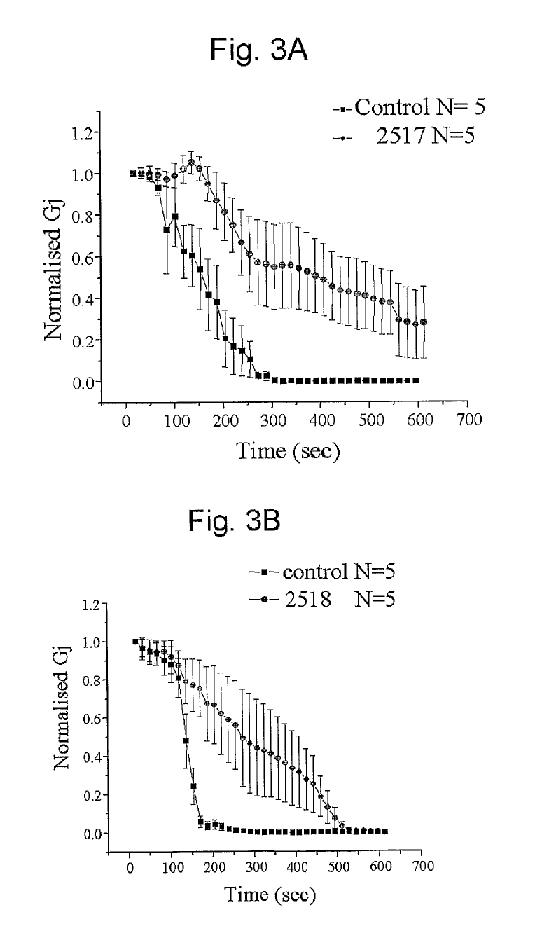

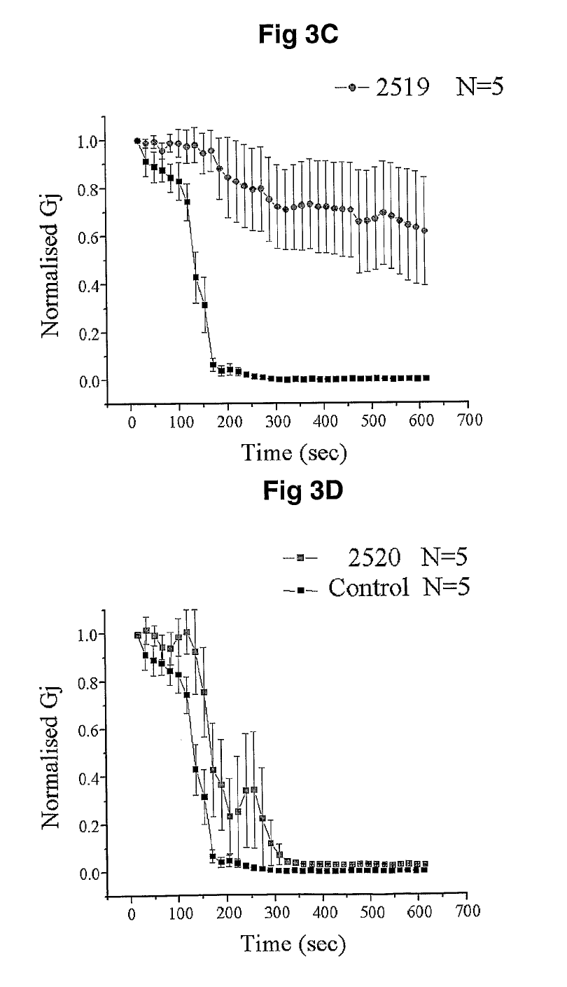

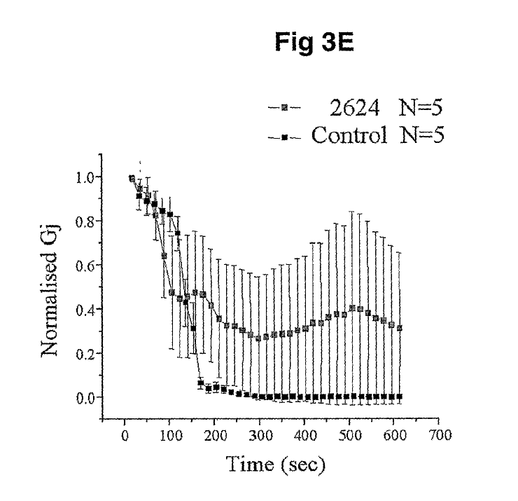

Experiments were modeled after those described in Shibayama et al (2006). Junctional conductance between Cx43-expressing N2a cell pairs was assessed using conventional dual patch clamp. Peptides were diluted in the patch pipette solution and the cells will be exposed either to octanol or to a low intracellular pH. Control experiments were carried out in the absence of the compound. The results are shown in FIGS. 3A-E, which are graphs of the time course of octanol-induced changes in coupling recorded from Cx43-expressing N2a cells. FIGS. 3A-E relate to peptides 2517, 2518, 2529, 2520 and 2624 respectively. The patch clamp experiments were conducted in the absence (black trace, square data points) or in the presence (red trace, rounded data points) of 0.1 mM of the peptides 2517, 2518, 2519, 2520 and 2624 in the internal pipette solution. Time zero corresponds to the onset of octanol superfusion. The relative decrease in junctional conductance was determined.

Uncoupling is defined by the total loss of junctional conductance. The number of cell pairs that uncouple when in the presence/absence of the peptide is established. Additional studies will be conducted to determine whether the effect of the peptides is dependent on the integrity of the Cx43CT domain.

### Example 8

### Binding of Peptides to Cx43CT

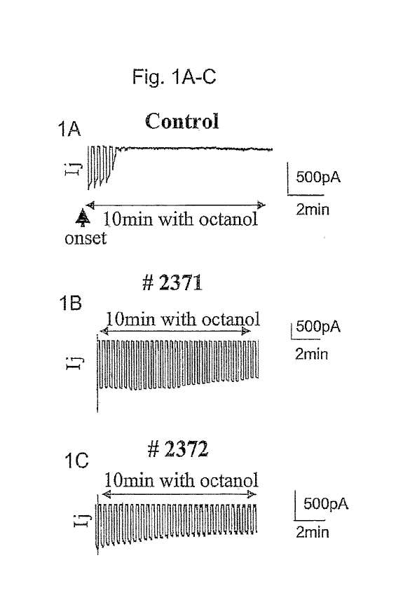

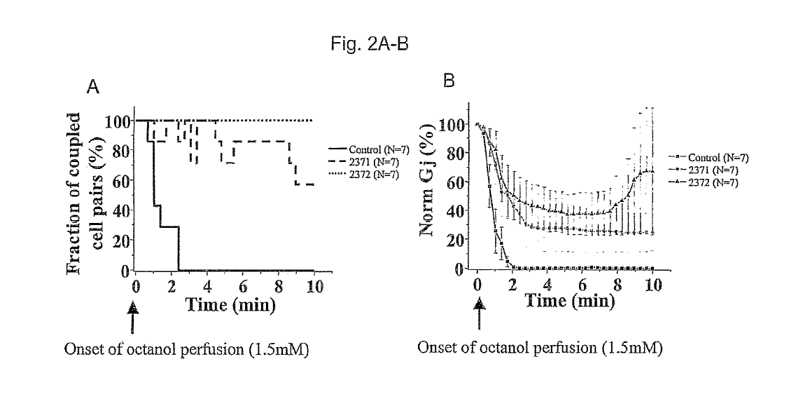

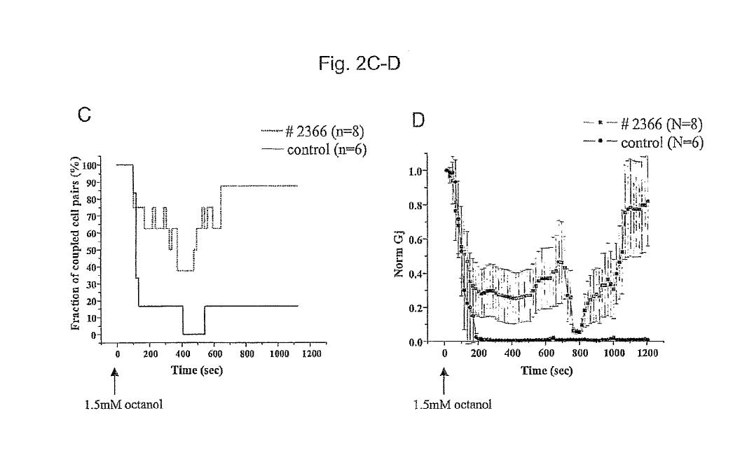

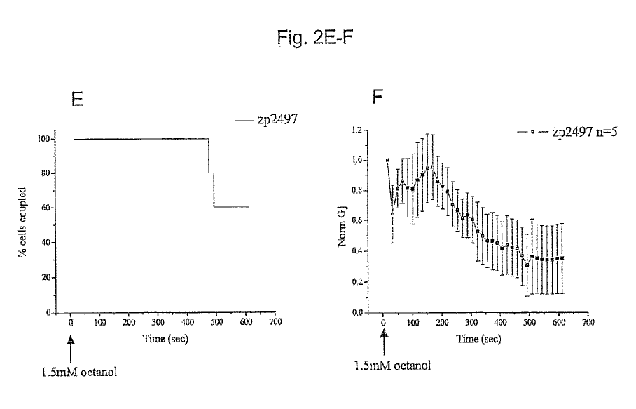

The ability of certain peptides in this application to bind to Cx43CT suggests that this peptide may also alter the behavior of Cx43 channels. Gap junction currents were recorded from N2a cells transfected with Cx43. To reduce macroscopic currents and allow for detection of single channel events, cell pairs were superfused with octanol (Anumonwo et al., “The Carboxyl Terminal Domain Regulates the Unitary Conductance and Voltage Dependence of Connexin40 Gap Junction Channels,” Circ. Res. 88(7):666-673 (2001); Seki et al., “Modifications in the Biophysical Properties of Connexin43 Channels by a Peptide of the Cytoplasmic Loop Region,” Circ. Res. 95(4):e22-e28 (2004); Seki et al., “Loss of Electrical Communication, but Not Plaque Formation, After Mutations in the Cytoplasmic Loop of Connexin43,” Heart Rhythm. 1(2):227-233 (2004), which are hereby incorporated by reference in their entirety). The presence of the certain peptides in this application in the internal pipette solution prevented octanol-induced closure of Cx43 channels, as shown in FIGS. 1A-C. FIG. 1A depicts junctional current traces obtained from a Cx43-expressing cell pair under control conditions (no peptides). Transjunctional voltage (V;) was +60 mV. Octanol was added to the superfusate at the point indicated by the arrow. After a short delay, junctional current abruptly decreased, reaching zero within three minutes after onset of octanol. The ability of octanol to uncouple gap junctions with a high degree of efficiency has been extensively reported in the literature (Johnston & Ramon, “Electrotonic Coupling in Internally Perfused Crayfish Segmented Axons,” J. Physiol. 317:509-518 (1981), which is hereby incorporated by reference in its entirety). The traces shown in FIGS. 1B and C were obtained from a different cell pair. Here, the peptides 2371 and 2372 were diluted in the internal pipette solution. Three minutes after onset of octanol, no or only a minor decrease in Gⱼ was observed. Cumulative data are presented in FIGS. 2A-F. FIGS. 2A, C and E depict the percent of cell pairs that remained coupled following the onset of octanol superfusion (uncoupling defined as zero junctional current elicited by a 60-mV transjunctional voltage pulse). Seven different cell pairs were recorded using patch pipettes filled with a solution containing of the peptides. Seven pairs were tested without peptides. Control experiments (no peptides) were conducted on cells in the same plate as those that failed to uncouple in the presence of the peptide. As shown in FIG. 1A, none of the cell pairs exposed to 2372 uncoupled following octanol (discontinuous line), whereas cell pairs are uncoupled and then recoupled during treatment with peptides 2371 and 2366 (1A and C). 6 out of 7 control cell pairs in absence of peptides (control, continuous line) showed complete uncoupling after 2 minutes of octanol superfusion. FIG. 1B shows the time course of changes in junctional conductance (Gⱼ; measured relative to control for each individual experiment) as a function of time after onset of octanol. Data obtained in the absence of peptides is depicted by a continuous line and closed symbols. The results show the characteristic rapid drop in reaching an asymptotic value after approximately 2 minutes of octanol superfusion. Broken lines correspond to data obtained in the presence of the peptides 2371, 2372, 2366 and 2497. Though no uncoupling was observed for the peptide 2372, a slight decrease in Gⱼ was apparent. Overall, the data show that all the compounds # 2371, 2372, 2366 and 2497 prevented octanol-induced uncoupling in Cx43-expressing cell pairs. Furthermore, 2366, 2371 and 2372 were able to revert uncoupled cells to couple cells The effect of the peptides are not ascribable to the presence of any peptidic molecule in the patch pipette. Indeed, previous studies have shown that a peptide derived from the cytoplasmic loop altered channel characteristics but did not alter octanol-sensitivity (Seki et al., “Modifications in the Biophysical Properties of Connexin43 Channels by a Peptide of the Cytoplasmic Loop Region,” Circ. Res. 95(4):e22-e28 (2004), which is hereby incorporated by reference in its entirety).

1. A peptide having Formula I:
R¹-Z-(L-Q)ₚ-R²
wherein
R¹ is selected from H, Ac, benzoyl and Tfa;
Z is A1-A2-A3-A4 or a retro analogue thereof, wherein:
A1 is a basic amino acid such as Arg, Lys or His or A1 is a lysine mimetic;
A2 is a basic amino acid such as Arg, Lys or His or A2 is a lysine mimetic;
A3 is any amino acid, preferably selected from Gly, Pro, Ala, Val, Leu, Ile, Met, Cys, Phe, Tyr, Trp, His, Lys, Arg, Gln, Asn, Glu, Asp, Ser and Thr, which amino acid is optionally modified with B, or A3 is a lysine mimetic, which lysine mimetic is optionally modified with B, or A3 is Glx(CONH-B);
A4 is an aromatic amino acid such as Trp, Tyr, Phe or His, or A4 is an aliphatic amino acid such as Ala, Val, Leu or Ile, or A4 is Met, or A4 is missing;
wherein B is a hydrophobic group;
L is X1-X2-X3-X4-X5-X6-X7-X8-X9-X10-X11-X12-X13-X14-X15 or a retro analogue thereof,
wherein
Xi is Gly, Ala, Ser, 8-amino-3,6-dioxaoctanic acid or is missing;
X2 is Gly, Ala, Ser, or is missing;
X3 is Gly, Ala, Ser, Leu, Val, Ile or is missing;
X4 is Gly, Ala, Ser, or is missing;
X5 is Gly, Ala, Ser, Arg, Lys, His or is missing;
X6 is Val, Ile, Leu, Gly, Ala, Ser, or is missing;
X7 is Pro, Gly, Ala, Ser, or is missing;
X10 is Gly, Ala, Ser, Arg, Lys, His or is missing;
X11 is Arg, Lys, His, Gly, Ala, Ser, or is missing;
X12 is Gly, Ala, Ser, or is missing;
X13 is Val, Leu, Ile or is missing;
X14 is Gly, Ala, Ser, or is missing;
X15 is Arg, Lys, His or is missing;
Q is A5-A6-A7-A8, or a retro analogue thereof, wherein
A5 is a basic amino acid such as Arg, Lys or His or A5 is a lysine mimetic;
A6 is a basic amino acid such as Arg, Lys or His or A6 is a lysine mimetic;
A7 is any amino acid, preferably selected from Gly, Pro, Ala, Val, Leu, Ile, Met, Cys, Phe, Tyr, Trp, His, Lys, Arg, Gin, Asn, Glu, Asp, Ser and Thr, which amino acid is optionally modified with B, or A7 is a lysine mimetic, which lysine mimetic is optionally modified with B, or A7 is Glx(CONH-B);
A8 is an aromatic amino acid such as Trp, Tyr, Phe or His, or A8 is an aliphatic amino acid such as Ala, Val, Leu or Ile, or A8 is Met, or A8 is missing;
R² is NH₂, OH, OR, NHR, NRR
wherein R is C₁-C₆ alkyl; and
p is 0, 1, 2, 3,4 or 5.

2. A peptide according to claim 1, wherein
when A4 is present, A3 is an amino acid selected from Gly, Pro, Ala, Val, Leu, Ile, Met, Cys, Phe, Tyr, Trp, His, Lys, Arg, Gln, Asn, Glu, Asp, Ser and Thr; and when A4 is missing, A3 is an amino acid selected from Gly, Pro, Ala, Val, Leu, Ile, Met, Cys, Phe, Tyr, Trp, His, Lys, Arg, Gln, Asn, Glu, Asp, Ser and Thr, which amino acid is modified with B, or A3 is a lysine mimetic, which lysine mimetic is modified with B, or A3 is Glx(CONH-B).

3. A peptide according to claim 1, wherein p=0, and wherein A1 is Lys, Arg or (2S4R)Amp;
A2 is Lys, Arg or (2S4R)Amp;
A3 is Lys(NH-B) or Asn(CONH-B) if A4 is missing, and A3 is Asn or Gln if A4 is present;
A4 is Trp, Tyr, Phe or His or is missing.

4. A peptide according to claim 1 wherein p=0.

5. A peptide according to claim 1 wherein p=1, 2, 3, 4 or 5, and wherein
when A8 is present, A7 is selected from Gly, Pro, Ala, Val, Leu, Ile, Met, Cys, Phe, Tyr, Trp, His, Lys, Arg, Gln, Asn, Glu, Asp, Ser and Thr; and
when A8 is missing, A7 is an amino acid selected from Gly, Pro, Ala, Val, Leu, Ile, Met, Cys, Phe, Tyr, Trp, His, Lys, Arg, Gln, Asn, Glu, Asp, Ser and Thr, which amino acid is modified with B, or A7 is a lysine mimetic, which lysine mimetic is modified with B, or A7 is Glx(CONH-B).

6. A peptide according to claim 1, wherein p=1, 2, 3, 4 or 5, and wherein
A 1 and A5 are the same amino acid or the same lysine mimetic;
A2 and A6 are the same amino acid or lysine mimetic;
A3 and A7 are the same amino acid or lysine mimetic, or are both Glx(NH-B);
A3 and A8 are the same amino acid or are both missing.

7. A peptide according to claim 1 wherein B comprises a 6- to 12-membered optionally substituted aromatic carbon ring.

8. A peptide according to claim 7 wherein B is an optionally substituted aralkyl, aryl or aroyl group comprising a 6- to 12-membered carbon aromatic ring.

9. A peptide according to claim 8 wherein B is benzyl, 4-hydroxybenzyl, benzoyl or 4-hydroxybenzoyl.

10. A peptide according to claim 1, selected from the group consisting of
Ac-Tyr-Asn-Arg-Arg-Gly-Gly-Gly-Ser-Ala-Val-Pro-Phe-Tyr-Ser-His-Ser-Tyr-Asn-Arg-Arg-NH2
Ac-Arg-Arg-Asn-Tyr-Arg-Arg-Asn-Tyr-NH2
Ac-Arg-Arg-Asn-Tyr-NH2
Ac-Tyr-Asn-Arg-Arg-NH2
Ac-Arg-Arg-Asn-Tyr-Arg-Asn-Pro-NH2
Ac-Arg-Arg-Asn-Tyr-Ser-Ala-Val-Pro-Phe-Tyr-Ser-His-Ser-Arg-Arg-Asn-Tyr-NH2
Ac-Arg-Arg-Asn-Tyr-Ser-His-Ser-Arg-Arg-Asn-Tyr-NH2
Ac-Arg-Arg-Asn-Tyr-Gly-Gly-Gly-Ser-Ala-Val-Pro-Phe-Tyr-Arg-Arg-Asn-Tyr-NH2
Ac-Arg-Arg-Asn-Tyr-Gly-Gly-Gly-Arg-Arg-Asn-Tyr-NH2
Ac-Arg-Arg-Asn-Tyr-Gly-Gly-Ser-Ala-Val-Pro-Phe-Tyr-Ser-His-Arg-Arg-Asn-Tyr-NH2
Ac-Arg-Arg-Asn-Tyr-Ala-Val-Pro-Phe-Arg-Arg-Asn-Tyr-NH2
Ac-Arg-Asn-Tyr-Val-Pro-Arg-Arg-Asn-Tyr-NH2
Ac-Arg-Arg-Asn-Tyr-Gly-Gly-Gly-Ser-Ala-Val-Pro-Phe-Tyr-Ser-His-Ser-Arg-Arg-Asn-Tyr-NH2
Asn-Tyr-NH2
Ac-Arg-Arg-Asn-Tyr-Ala-Ala-Leu-Ala-Lys-Ala-Ala-Leu-Ala-Lys-Ala-Ala-Leu-Ala-Lys-Arg-Arg-Asn-Tyr-NH2
Ac-Arg-Arg-Asn-Tyr-(8-amino-3,6-dioxaoctanoic acid)-Arg-Arg-Asn-Tyr-NH2
Ac-Arg-Arg-Asn-Tyr-Gly-Gly-Gly-Ser-Ala-Val-Pro-Phe-Tyr-Ser-His-Ser-Arg-Arg-Asn-Tyr-Gly-Gly-Gly-Ser-Ala-Val-Pro-Phe-Tyr-Ser-His-Ser-Arg-Arg-Asn-Tyr-NH2
Ac-Arg-Arg-Asn-Tyr-Gly-Gly-Gly-Ser-Ala-Val-Pro-Phe-Tyr-Ser-His-Ser-Arg-Arg-Asn-Tyr-Gly-Gly-Gly-Ser-Ala-Val-Pro-Phe-Tyr-Ser-His-Ser-Arg-Arg-Asn-Tyr-Gly-Gly-Gly-Ser-Ala-Val-Pro-Phe-Tyr-Ser-His-Ser-Arg-Arg-Asn-Tyr-NH2
H-(2S ,4R)Amp((2S,4R)Amp(Ac))-Asn-Tyr-NH2
Ac-Arg-Arg-Asn(CONH-Bzl)-NH2
Ac-Arg-Arg-Lys (NH-4-hydroxybenzoyl)-NH2
Ac-Lys(NH-4-hydroxybenzyol)Arg-Arg-NH2
and pharmaceutically acceptable salts thereof.

11. A peptide according to claim 1 for use in a method of medical treatment.

12. A peptide according to claim 11, for use in a method of medical treatment for treating a pathological condition involving impaired gap junctional communication.

13. The peptide according to claim 12, wherein the pathological condition is selected from the group consisting of a cardiovascular disease, inflammation of airway epithelium, a disorder of alveolar tissue, bladder incontinence, impaired disease, failure of bone marrow transplantation, wound, erectile dysfunction, urinary bladder incontinence, neuropathic pain, subchronic and chronic inflammation, cancer, transplantation failure; a condition caused by an excess of reactive oxygen species and/or free radicals and/or nitric oxide.

14. A peptide according to claim 1, wherein the peptide is a modulator of the function of the tissue, cell, or cell fraction.

15. A method for modulating gap junctional communication in a population of cells comprising administering an effective amount of a peptide according to claim 1 to the population of cells, thereby modulating gap junctional communication between the cells.

16. The method according to claim 15, wherein administering is performed in vivo.

17. A method of treating a patient having, or at risk of developing, a pathological condition involving impaired gap junctional communication comprising administering to the patient a therapeutically effective amount of a peptide according to claim 1.

18. The method according to claim 17 wherein the patient is a human being.

19. The method according to claim 17, where the pathological condition is selected from the group consisting of a cardiovascular disease, inflammation of airway epithelium, a disorder of alveolar tissue, bladder incontinence, impaired hearing, an endothelial lesion, diabetic retinopathy, diabetic neuropathy, ischemia of the central nervous system, ischemia of the spinal cord, a dental tissue disorder, kidney disease, failure of bone marrow transplantation, wound, erectile dysfunction, urinary bladder incontinence, neuropathic pain, subchronic and chronic inflammation, cancer, transplantation failure; a condition caused by an excess of reactive oxygen species and/or free radicals and/or nitric oxide, diabetes, osteoporosis and psoriasis.

20-22. (canceled)

23. A pharmaceutical composition comprising the peptide of claim 1 and a pharmaceutical carrier.


SEQUENCE LISTING (93 sequences)

SEQ ID NO: 1 - protein, 4 aa, Artificial sequence
  Synthetic sequence
KKNF

SEQ ID NO: 2 - protein, 4 aa, Artificial sequence
  Synthetic sequence
KKNY

SEQ ID NO: 3 - protein, 4 aa, Artificial sequence
  Synthetic sequence
KKNH

SEQ ID NO: 4 - protein, 4 aa, Artificial sequence
  Synthetic sequence
KKNW

SEQ ID NO: 5 - protein, 4 aa, Artificial sequence
  Synthetic sequence
KKNV

SEQ ID NO: 6 - protein, 4 aa, Artificial sequence
  Synthetic sequence
KKNL

SEQ ID NO: 7 - protein, 4 aa, Artificial sequence
  Synthetic sequence
KKNI

SEQ ID NO: 8 - protein, 4 aa, Artificial sequence
  Synthetic sequence
KKNM

SEQ ID NO: 9 - protein, 4 aa, Artificial sequence
  Synthetic sequence
RRNH

SEQ ID NO: 10 - protein, 4 aa, Artificial sequence
  Synthetic sequence
RRNW

SEQ ID NO: 11 - protein, 4 aa, Artificial sequence
  Synthetic sequence
RRNI

SEQ ID NO: 12 - protein, 4 aa, Artificial sequence
  Synthetic sequence
RRNM

SEQ ID NO: 13 - protein, 4 aa, Artificial sequence
  Synthetic sequence
RRNY

SEQ ID NO: 14 - protein, 4 aa, Artificial sequence
  Synthetic sequence
RRNV

SEQ ID NO: 15 - protein, 4 aa, Artificial sequence
  Synthetic sequence
RRNL

SEQ ID NO: 16 - protein, 4 aa, Artificial sequence
  Synthetic sequence
RRNF

SEQ ID NO: 17 - protein, 4 aa, Artificial sequence
  Synthetic sequence
RRNY

SEQ ID NO: 18 - protein, 4 aa, Artificial sequence
  Synthetic sequence
RRNH

SEQ ID NO: 19 - protein, 4 aa, Artificial sequence
  Synthetic sequence
RRNW

SEQ ID NO: 20 - protein, 4 aa, Artificial sequence
  Synthetic sequence
RRNF

SEQ ID NO: 21 - protein, 4 aa, Artificial sequence
  Synthetic sequence
RRNI

SEQ ID NO: 22 - protein, 4 aa, Artificial sequence
  Synthetic sequence
RRNM

SEQ ID NO: 23 - protein, 4 aa, Artificial sequence
  Synthetic sequence
RRNV

SEQ ID NO: 24 - protein, 4 aa, Artificial sequence
  Synthetic sequence
RRNL

SEQ ID NO: 25 - protein, 4 aa, Artificial sequence
  Synthetic sequence
YNRR

SEQ ID NO: 26 - protein, 4 aa, Artificial sequence
  Synthetic sequence
YNKR

SEQ ID NO: 27 - protein, 4 aa, Artificial sequence
  Synthetic sequence
YNRK

SEQ ID NO: 28 - protein, 4 aa, Artificial sequence
  Synthetic sequence
YNKK

SEQ ID NO: 29 - protein, 4 aa, Artificial sequence
  Synthetic sequence
YNRR

SEQ ID NO: 30 - protein, 4 aa, Artificial sequence
  Synthetic sequence
YNKR

SEQ ID NO: 31 - protein, 4 aa, Artificial sequence
  Synthetic sequence
YNRK

SEQ ID NO: 32 - protein, 4 aa, Artificial sequence
  Synthetic sequence
YNKK

SEQ ID NO: 33 - protein, 4 aa, Artificial sequence
  Synthetic sequence
KKQF

SEQ ID NO: 34 - protein, 4 aa, Artificial sequence
  Synthetic sequence
KKQY

SEQ ID NO: 35 - protein, 4 aa, Artificial sequence
  Synthetic sequence
KKQH

SEQ ID NO: 36 - protein, 4 aa, Artificial sequence
  Synthetic sequence
KKQW

SEQ ID NO: 37 - protein, 4 aa, Artificial sequence
  Synthetic sequence
KKQV

SEQ ID NO: 38 - protein, 4 aa, Artificial sequence
  Synthetic sequence
KKQL

SEQ ID NO: 39 - protein, 4 aa, Artificial sequence
  Synthetic sequence
KKQI

SEQ ID NO: 40 - protein, 4 aa, Artificial sequence
  Synthetic sequence
KKQM

SEQ ID NO: 41 - protein, 4 aa, Artificial sequence
  Synthetic sequence
RRQH

SEQ ID NO: 42 - protein, 4 aa, Artificial sequence
  Synthetic sequence
RRQW

SEQ ID NO: 43 - protein, 4 aa, Artificial sequence
  Synthetic sequence
RRQI

SEQ ID NO: 44 - protein, 4 aa, Artificial sequence
  Synthetic sequence
RRQM

SEQ ID NO: 45 - protein, 4 aa, Artificial sequence
  Synthetic sequence
RRQY

SEQ ID NO: 46 - protein, 4 aa, Artificial sequence
  Synthetic sequence
RRQV

SEQ ID NO: 47 - protein, 4 aa, Artificial sequence
  Synthetic sequence
RRQL

SEQ ID NO: 48 - protein, 4 aa, Artificial sequence
  Synthetic sequence
RRQF

SEQ ID NO: 49 - protein, 4 aa, Artificial sequence
  Synthetic sequence
XXNF

SEQ ID NO: 50 - protein, 4 aa, Artificial sequence
  Synthetic sequence
XXNY

SEQ ID NO: 51 - protein, 4 aa, Artificial sequence
  Synthetic sequence
XXNH

SEQ ID NO: 52 - protein, 4 aa, Artificial sequence
  Synthetic sequence
XXNW

SEQ ID NO: 53 - protein, 4 aa, Artificial sequence
  Synthetic sequence
XXNV

SEQ ID NO: 54 - protein, 4 aa, Artificial sequence
  Synthetic sequence
XXNL

SEQ ID NO: 55 - protein, 4 aa, Artificial sequence
  Synthetic sequence
XXNI

SEQ ID NO: 56 - protein, 4 aa, Artificial sequence
  Synthetic sequence
XXNM

SEQ ID NO: 57 - protein, 4 aa, Artificial sequence
  Synthetic sequence
RKNF

SEQ ID NO: 58 - protein, 4 aa, Artificial sequence
  Synthetic sequence
RKNY

SEQ ID NO: 59 - protein, 4 aa, Artificial sequence
  Synthetic sequence
RKNH

SEQ ID NO: 60 - protein, 4 aa, Artificial sequence
  Synthetic sequence
RKNW

SEQ ID NO: 61 - protein, 4 aa, Artificial sequence
  Synthetic sequence
RKNV

SEQ ID NO: 62 - protein, 4 aa, Artificial sequence
  Synthetic sequence
RKNL

SEQ ID NO: 63 - protein, 4 aa, Artificial sequence
  Synthetic sequence
RKNI

SEQ ID NO: 64 - protein, 4 aa, Artificial sequence
  Synthetic sequence
RKNM

SEQ ID NO: 65 - protein, 4 aa, Artificial sequence
  Synthetic sequence
KRNF

SEQ ID NO: 66 - protein, 4 aa, Artificial sequence
  Synthetic sequence
KRNY

SEQ ID NO: 67 - protein, 4 aa, Artificial sequence
  Synthetic sequence
KRNH

SEQ ID NO: 68 - protein, 4 aa, Artificial sequence
  Synthetic sequence
KRNW

SEQ ID NO: 69 - protein, 4 aa, Artificial sequence
  Synthetic sequence
KRNV

SEQ ID NO: 70 - protein, 4 aa, Artificial sequence
  Synthetic sequence
KRNL

SEQ ID NO: 71 - protein, 4 aa, Artificial sequence
  Synthetic sequence
KRNI

SEQ ID NO: 72 - protein, 4 aa, Artificial sequence
  Synthetic sequence
KRNM

SEQ ID NO: 73 - protein, 8 aa, Artificial sequence
  Synthetic sequence
KKNYKKNY

SEQ ID NO: 74 - protein, 8 aa, Artificial sequence
  Synthetic sequence
RRNYRRNY

SEQ ID NO: 75 - protein, 8 aa, Artificial sequence
  Synthetic sequence
RKNYRKNY

SEQ ID NO: 76 - protein, 8 aa, Artificial sequence
  Synthetic sequence
RKNYKRNY

SEQ ID NO: 77 - protein, 8 aa, Artificial sequence
  Synthetic sequence
RRNYKKNY

SEQ ID NO: 78 - protein, 8 aa, Artificial sequence
  Synthetic sequence
KKNYRRNY

SEQ ID NO: 79 - protein, 20 aa, Artificial sequence
  Synthetic sequence
YNRRGGGSAVPFYSHSYNRR

SEQ ID NO: 80 - protein, 8 aa, Artificial sequence
  Synthetic sequence
RRNYRRNY

SEQ ID NO: 81 - protein, 17 aa, Artificial sequence
  Synthetic sequence
RRNYSAVPFYSHSRRNY

SEQ ID NO: 82 - protein, 11 aa, Artificial sequence
  Synthetic sequence
RRNYSHSRRNY

SEQ ID NO: 83 - protein, 17 aa, Artificial sequence
  Synthetic sequence
RRNYGGGSAVPFYRRNY

SEQ ID NO: 84 - protein, 11 aa, Artificial sequence
  Synthetic sequence
RRNYGGGRRNY

SEQ ID NO: 85 - protein, 18 aa, Artificial sequence
  Synthetic sequence
RRNYGGSAVPFYSHRRNY

SEQ ID NO: 86 - protein, 12 aa, Artificial sequence
  Synthetic sequence
RRNYAVPFRRNY

SEQ ID NO: 87 - protein, 10 aa, Artificial sequence
  Synthetic sequence
RRNYVPRRNY

SEQ ID NO: 88 - protein, 20 aa, Artificial sequence
  Synthetic sequence
RRNYGGGSAVPFYSHSRRNY

SEQ ID NO: 89 - protein, 23 aa, Artificial sequence
  Synthetic sequence
RRNYAALAKAALAKAALAKRRNY

SEQ ID NO: 90 - protein, 9 aa, Artificial sequence
  Synthetic sequence
RRNYXRRNY

SEQ ID NO: 91 - protein, 36 aa, Artificial sequence
  Synthetic sequence
RRNYGGGSAVPFYSHSRRNYGGGSAVPFYSHSRRNY

SEQ ID NO: 92 - protein, 52 aa, Artificial sequence
  Synthetic sequence
RRNYGGGSAVPFYSHSRRNYGGGSAVPFYSHSRRNYGGGSAVPFYSHSRRNY

SEQ ID NO: 93 - protein, 7 aa, Artificial sequence
  Synthetic sequence
RRNYRNP